SEQUENCE LISTING (6 sequences)

SEQ ID NO: 1 - protein, 12 aa, Artificial Sequence
  Tat
YGRKKKRRQRRR

SEQ ID NO: 2 - protein, 18 aa, Artificial Sequence
  pVEC
LLIILRRRIRKQAHAHSK

SEQ ID NO: 3 - protein, 18 aa, Artificial Sequence
  RRL helix
RRLRRLLRRLRRLLRRLR

SEQ ID NO: 4 - protein, 16 aa, Artificial Sequence
  shuffle
RWFKIQMQIRRWKNKK

SEQ ID NO: 5 - protein, 16 aa, Artificial Sequence
  penetramax
KWFKIQMQIRRWKNKR

SEQ ID NO: 6 - protein, 23 aa, Artificial Sequence
  antimicrobial peptide
GIGKFLHSAKKFGKAFVGEIMNS

(19) United States
(12) Patent Application Publication
(10) Pub. No.: US 2024/0074970 A1
(43) Pub. Date: Mar. 7, 2024

(54) COMPOSITIONS FOR THE DELIVERY OF PROTEINS

(71) Applicant: Afyx Therapeutics A/S, København S (DK)

(72) Inventors: Martin Eduardo Santocildes ROMERO, København S (DK); Jake G. EDMANS, København S (DK)

(21) Appl. No.: 17/767,387

(22) PCT Filed: Oct. 8, 2020
(86) PCT No.: PCT/US2020/054832

§ 371 Date: Apr. 7, 2022

Related U.S. Application Data
(60) Provisional application No. 62/912,377, filed on Oct. 8, 2019.

(51) Int. Cl. A61K 9/00; A61K 38/04; A61K 38/28; A61K 38/46; A61K 47/12; A61K 47/32; A61K 47/34; A61K 47/40; A61K 47/42

(52) U.S. Cl. CPC A61K 9/006; A61K 38/043; A61K 38/28; A61K 38/465; A61K 47/12; A61K 47/32; A61K 47/34; A61K 47/40; A61K 47/42

(57) ABSTRACT

The present disclosure relates to electrospun patches comprising therapeutic polypeptides. The patches include an impermeable layer that functions as a barrier, e.g., to prevent to penetration of water. Upon attachment of the patch to skin or mucosa, the therapeutic polypeptide is released in order to treat a variety of diseases and conditions.

### CROSS-REFERENCE TO RELATED APPLICATIONS

This application claims the benefit of and priority to U.S. Provisional Application No. 62/912,377 filed Oct. 8, 2019, which is hereby incorporated by reference in its entirety.

### TECHNICAL FIELD

The present disclosure, without limitation and in various embodiments, relates to mucoadhesive patches comprising an electrospun layer and an impermeable layer. The electrospun layer includes one or more polymers and a therapeutic polypeptide, e.g., an antibody, protein, peptide or enzyme, which is controllably released and delivered to the oral mucosa at a clinically effective rate. The patches disclosed herein are therefore suitable for a range of applications including the treatment of resistant infections, tissue regeneration, and as an alternative to injections for systemic delivery. The present disclosure, in other embodiments, relates to particular electrospun fibers, methods of use of such fibers and patches, and methods of manufacturing the electrospun fibers and patches disclosed herein.

### BACKGROUND

A variety of potential applications for biopharmaceutical delivery to the oral mucosa have been identified, such as the use of antimicrobial peptides as a treatment for resistant bacterial and fungal infections, the use of growth factors to regenerate tissue, and as an alternative to injections for systemic delivery. However, existing formulations (e.g., mouthwashes, gels, tablets, and dissolvable films) targeting the oral mucosa typically afford non-specific release of the active ingredient, rather than local release to a specific region where drug is required. In addition, these formulations provide little control over dose, and therefore are often ineffective.

Accordingly, there is a need in the art for new delivery systems that provide controlled release of a therapeutic polypeptide at a targeted site (e.g., the oral mucosa) and provide sufficient exposure of the active agent in order to effectively treat diseases and conditions of interest.

### SUMMARY

To address the aforementioned issues, the present disclosure provides electrospun mucoadhesive patches with an impermeable layer that are designed for localized, unidirectional delivery of therapeutic polypeptides to a targeted site, e.g., mucosa or skin. The disclosed patches offer advantages over existing alternatives such as high encapsulation efficiency and fast release rates of the active agent, long residence times at the treatment site, high patient acceptability, and high surface area.

Accordingly, the patches disclosed herein are useful in treating a variety diseases and conditions, including those that affect the oral mucosa.

In one aspect, the present disclosure provides a patch comprising:

(a) an electrospun fiber layer, wherein the electrospun fibers comprise: one or more polymers and a therapeutically effective amount of a therapeutic polypeptide; and (b) an impermeable layer.

In one aspect, the present disclosure provides a patch comprising an electrospun fiber layer, wherein the electrospun fibers comprise: one or more polymers and a therapeutically effective amount of a therapeutic polypeptide.

In some embodiments, the electrospun fibers are uniaxial.

In some embodiments, the electrospun fiber layer comprises about 0.01% to about 50.0% (wt./wt. %) of the therapeutic polypeptide. In some embodiments, the electrospun fiber layer comprises about 1.0% to about 10.0% (wt./wt. %) of the therapeutic polypeptide

In some embodiments, the therapeutic polypeptide is selected from the group consisting of a monoclonal antibody, a fragment antigen-binding (Fab fragment) antibody, a single-chain variable fragment (scFv), a full antigen, a fragmented antigen, a protein, a peptide, and an enzyme.

In some embodiments, the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the impermeable layer is hydrophobic. In some embodiments, the hydrophobic impermeable layer comprises polycaprolactone.

In some embodiments, the electrospun fibers further comprise an absorption enhancer. In some embodiments, the absorption enhancer is selected from the group consisting of a fatty acid, a non-ionic surfactant, a polycation, a thiolated polymer, a cyclodextrin, and a cell-penetrating peptide.

In one aspect, the present disclosure provides a patch comprising: (a) an electrospun fiber layer, wherein the electrospun fibers comprise: one or more polymers, a therapeutically effective amount of a therapeutic polypeptide, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a protein, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a peptide, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a monoclonal antibody, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a fragment antigen-binding (Fab fragment) antibody, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a single-chain variable fragment, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of a full antigen or a fragmented antigen, and an absorption enhancer; and (b) a hydrophobic impermeable layer.

In one aspect, the present disclosure provides a patch comprising: (a) an electrospun fiber layer, wherein the electrospun fibers comprise: one or more polymers, a therapeutically effective amount of an enzyme, and an absorption enhancer; and (b) a hydrophobic impermeable layer.

In some embodiments, the patch provides a therapeutically effective amount of the therapeutic polypeptide for at least about 4 h following application to a patient in need thereof

In one aspect, the present disclosure provides methods of treating a condition in a patient in need thereof, the method comprising administering a patch disclosed herein, wherein the condition is selected from the group consisting of recurrent aphthous stomatitis, oral lichen planus, pemphigoid, pemphigus, oral mucositis, Graft versus host disease (GvHD), Bechet's disease, lupus erythematosus, vulva lichen planus, Lipschutz ulcers, lichen simplex chronicus, vulva psoriasis, allergies, autoimmune disorders or disorders with an immune component (multiple sclerosis, rheumatoid arthritis), osteoporosis, inflammatory bowel disease, hypercholesterolemia, asthma, hematologic malignancies, solid tumors, allograft rejection, and infectious organisms.

In one aspect, the present disclosure provides a method of preparing a patch, the process comprising (a) combining an aqueous alcoholic solvent with one or more polymers and a therapeutic polypeptide to provide an electrospinning mixture; (b) electrospinning the electrospinning mixture of step (a) to provide electrospun fibers; and (c) attaching an impermeable backing layer to the electrospun fibers of step (b).

### BRIEF DESCRIPTION OF THE DRAWINGS

FIG. 1A shows the conductivity of electrospinning solutions containing lysozyme with different mixtures of ethanol and water as solvents and placebo solutions in 97 v/v % ethanol without lysozyme (P). Data are presented as mean±SD, with three independent repeats and analyzed using one-way ANOVA with post hoc Tukey tests. *, p<0.05; **, p<0.01; ***, P<0.001.

FIG. 1B shows the average viscosity of electrospinning solutions containing lysozyme with different mixtures of ethanol and water as solvents and placebo solutions in 97 v/v % ethanol without lysozyme (P) measured at shear rates 0.1-100 s⁻¹. Data are presented as mean±SD, with three independent repeats and analyzed using one-way ANOVA with post hoc Tukey tests. *, p<0.05; **, p<0.01; ***, P<0.001.

FIGS. 2A-2E provide scanning electron micrographs of placebo fibers (FIG. 2A) and electrospun fibers comprising lysozyme manufactured using: 97% v/v % ethanol (FIG. 2B), 80% v/v % ethanol (FIG. 2C), 60% v/v % ethanol (FIG. 2D), and 40% v/v % ethanol (FIG. 2E).

FIG. 2F shows a graph of fiber diameter distributions for each of the electrospun fibers in FIGS. 2A-2E. Data are presented as median, interquartile range, and range with three independently prepared samples for each solvent mixture and ten diameter measurements per sample and analyzed using one-way ANOVA with post hoc Tukey tests. *, p<0.05; ***, P<0.001.

FIG. 3A provides a scanning electron micrograph of PVP and RS100 electrospun fibers comprising 0.1% w/w bradykinin.

FIG. 3B shows a graph of fiber diameter distributions for placebo fibers (no bradykinin) and the fibers of FIG. 3A. Data are presented as median, interquartile range, and range with three independently prepared samples for each solvent mixture and ten diameter measurements per sample and analyzed using one-way ANOVA with post hoc Tukey tests. *, p<0.05; ***, P<0.001.

FIGS. 4A-4D provide scanning electron micrographs of PVP and RS100 electrospun fibers comprising 0% w/w insulin (FIG. 4A, placebo), 1% w/w insulin (FIG. 4B), 3% w/w insulin (FIG. 4C), 5% w/w insulin (FIG. 4D). Electrospun fibers were manufactured from a solution comprising 10% w/w PVP, 12.5% w/w RS100, and 80% v/v ethanol/20% v/v 2% acetic acid in PBS.

FIG. 4E shows a graph of fiber diameter distributions for placebo fibers (no insulin) and fibers electrospun with 1%, 3%, and 5% insulin. Data are presented as median, interquartile range, and range with three independently prepared samples for each solvent mixture and ten diameter measurements per sample and analyzed using one-way ANOVA with post hoc Tukey tests. *, p<0.05; ***, P<0.001.

FIG. 5 shows a graph of the degree of swelling in water of lysozyme-containing electrospun fibers using different ethanol concentrations and placebo electrospun fibers (P). Data are presented as mean±SD, with 3 independent samples and analysed using one-way ANOVA with post hoc Tukey tests. Note: for 60 v/v % ethanol only 2 independent measurements were made, therefore SD was not calculated.

FIG. 6 provides a graph of encapsulation efficiency and activity of lysozyme in fibers electrospun using different mixtures of ethanol and water as solvents. Data is presented as mean±SD, with 3 independent samples per solvent mixture, and analyzed using one-way ANOVA with post hoc Tukey tests.

FIGS. 7A-7C show confocal micrographs of electrospun fibers containing FITC-PVP complex and Texas-red conjugated lysozyme to show the distribution of PVP (FIG. 7A, green) and lysozyme (FIG. 7B, red) within the fibers, along with an overlay of both distributions (FIG. 7C). Electrospun fibers were manufactured from 97 v/v % ethanol.

FIG. 7D provides a graph of encapsulation efficiency (EE) and activity of central, intermediate, and outer regions of lysozyme-containing fibers electrospun from 97 v/v % ethanol. Data is presented as mean±SD, with three independent samples for each region, and analyzed using one-way ANOVA with post hoc Tukey tests.

FIG. 8 shows the cumulative percent release of lysozyme from fibers electrospun using 97 v/v % ethanol as a solvent following immersion in PBS. Data is presented as mean±SD, with three independent samples for each time point.

FIG. 9A shows the increase in fluorescence emission determined by ELISA assay at various timepoints in cell monolayers exposed to bradykinin released from electrospun patches (0.1% w/w) immersed in phosphate-buffered saline (PBS). This is compared to the effect of placebo patches (i.e., patches without bradykinin) processed under the same conditions, where no fluorescence emission was detected.

FIG. 9B shows a quantitative comparison of the increase in fluorescence determined by ELISA assay evaluating patches containing bradykinin (BP), bradykinin dissolved in PBS (BK), placebo patches (PP) and pure phosphate-buffered saline (PBS).

FIG. 10 shows the percent release of bradykinin from the fibers of FIG. 3A following immersion in PBS. Data is presented as mean±SD, with three independent samples for each time point.

FIG. 11A and FIG. 11B show the cumulative release and percent cumulative release, respectively, of 1%, 3%, and 5% w/w insulin from fibers electrospun using 80% v/v % ethanol as a solvent following immersion in PBS. Data is presented as mean±SD, with three independent samples for each time point and compared to placebo (fibers without insulin).

FIG. 12A and FIG. 12B provide SEM micrographs of a continuous PCL backing layer formed after thermal treatment (FIG. 12A) and the edge of the patch showing the PCL film backing layer and lower layer of lysozyme-containing PVP and RS100 fibers (FIG. 12B).

FIG. 12C shows the percent activity of lysozyme released from electrospun patches comprising PVP and RS100 fibers with PCL backing layers before and after melting at 65° C. for 15 minutes. Data is presented as mean±SD, with three independent samples, and analyzed using Welch's t-test.

FIG. 13A provides a growth curve of S. ratti measured by optical density at 600 nm over time in the presence of PBS, eluent from placebo electrospun fibers (P), eluent from electrospun fibers containing lysozyme (LP), placebo fibers eluted in stock lysozyme solution (P+L), and a lysozyme stock solution (L). % growth inhibition relative to PBS at 15 h (B). Data is presented as mean±SD, with three independent samples, and the optical densities at 15 h analyzed using one-way ANOVA with post hoc Tukey tests. ****, p<0.0001.

FIG. 13B provides percent growth inhibition of S. ratti by eluent from placebo electrospun fibers (P), eluent from electrospun fibers containing lysozyme (LP), placebo electrospun fibers eluted in stock lysozyme solution (P+L), and a lysozyme stock solution (L), relative to PBS at 15 h. Data is presented as mean±SD, with three independent samples, and the optical densities at 15 h analyzed using one-way ANOVA with post hoc Tukey tests. ****, p<0.0001.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

“Optional” or “optionally” means that the subsequently described element, event or circumstances may or may not occur, and that the description includes instances where said event, element, or circumstance occurs and instances in which it does not.

As used herein, “patient” include vertebrates, more specifically a mammal (e.g., a human, horse, pig, rabbit, dog, sheep, goat, non-human primate, cow, cat, guinea pig or rodent), a fish, a bird or a reptile or an amphibian. In some embodiments, the patient is a human patient. Unless otherwise specified, the term patient does not denote a particular age or sex. In some embodiments, the patient may be a geriatric patient, a pediatric patient, a teenage patient, a young adult patient, or a middle-aged patient. In some embodiments, the patient is less than about 18 years of age. In some embodiments, the patient is at least about 18 years of age. In some embodiments, the patient is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age. In some embodiments the patient is a male patient. In some embodiments the patient is a female patient. In some embodiments, the patient is diagnosed with or is considered at risk of having or developing an infection, e.g., a bacterial, viral, or fungal infection.

As used herein, the term “impermeable layer” or “impermeable backing layer” refers to a coating or barrier layer in a layered patch configuration that prevents or substantially limits the passage of water through the layer. The impermeable layer can also prevent or substantially limit the passage of the active ingredient(s) (e.g., therapeutic polypeptide) through the layer.

As used herein, the term “absorption enhancer” or “permeation enhancer” refers to an additive in a patch or electrospun fibers that improves the ability of the therapeutic polypeptide to be absorbed by a targeted tissue or membrane, e.g., oral mucosa or skin.

As used herein, the term “treating” with regard to a patient, refers to improving at least one symptom of the patient's disorder. Treating can be curing, improving, or at least partially ameliorating a disorder. The term “therapeutic effect” as used herein refers to a desired or beneficial effect provided by the method and/or the composition. For example, the method for treating a disclosed disease or condition provides a therapeutic effect when the method reduces at least one symptom of the disease or condition in a patient.

As used herein, the term “preventing” with regard to a patient refers to reducing or eliminating the onset of the symptoms or complications of a disease, condition or disorder. In some embodiments, the symptoms or complications are reduced or eliminated in a patient that is predisposed to the disease, condition, or disorder. The patches of the present disclosure are capable of preventing or treating such diseases, condition, or disorders.

All documents cited herein are incorporated by reference in their entirety for all purposes.

### DETAILED DESCRIPTION

The oral mucosa is of interest for systemic delivery of therapeutic polypeptides, circumventing proteolytic degradation in the gastrointestinal tract and offering the potential for controlled release and needleless delivery. However, a significant challenge for the delivery of such active agents to the oral mucosa is the lack of suitable formulations that allow specific delivery. The most commonly used formulations targeting the oral mucosa include mouthwashes, gels, tablets, and dissolvable films. These drug delivery systems can be used with highly permeable drugs, but release of the active ingredient is often non-specific, i.e., across the entire oral cavity rather than localized into a specific region of the oral mucosa where the drug is required. Variations in salivary flow and mechanical forces mean that doses are poorly defined where prolonged contact is required.

Highly mucoadhesive oral patches that attach to mucosal surfaces and effectively deliver an active agent, e.g., the anesthetic lidocaine or the steroid clobetasol propionate, are described in International Publication Nos. WO2015/189212, WO2017/085264, WO2018/133910, and WO2018/133909; U.S. Pat. No. 10,052,291; and U.S. Publication Nos. 2019/0254985, 2019/0254986, and 2019/0351662, which are hereby incorporated by reference in their entireties for all purposes.

The present disclosure provides polypeptide-containing patches that are suitable for application to the oral mucosa. As described herein, the disclosed patches adhere to the oral mucosa and controllably release the active agent (i.e., a therapeutic polypeptide) in a localized manner at a clinically effective rate. The patches disclosed herein are therefore suitable for a range of applications including the treatment of resistant infections, tissue regeneration, and as an alternative to injections for systemic delivery

### Patches of the Present Disclosure

In some embodiments, the present disclosure provides a polypeptide-containing patch comprising: (a) an electrospun fiber layer, wherein the electrospun fibers comprise: one or more polymers and a therapeutically effective amount of a therapeutic polypeptide; and (b) an impermeable backing layer. The electrospun fiber layer and impermeable layer are joined to provide a patch using methods that are known to those skilled in the art, for example, the methods disclosed in International Publication No. WO/2018/133909.

In order to provide effective treatment, the patches of the present disclosure adhere to the targeted site (e.g., tissue, mucosa, skin, etc.) for a period of time sufficient to provide (i.e., release) an effective amount of a therapeutic polypeptide. Accordingly, in some embodiments, the patch adheres to the targeted site for a period greater than 2 h, greater than 4 h, greater than 8 h, greater than 12 h, greater than 16 h, greater than 24 h, greater than 30 h, greater than 36 h, greater than 42 h, or greater than 48 h. In some embodiments, the patch adheres to the targeted site for a period of at least 2 h. In some embodiments, the patch adheres to the targeted site for a period of at least 4 h. In some embodiments, the patch adheres to the targeted site for a period of at least 6 h. In some embodiments, the patch adheres to the targeted site for a period of at least 8 h. In some embodiments, the patch adheres to the targeted site for a period of at least 12 h. In some embodiments, the patch adheres to the targeted site for a period of at least 24 h.

Without being bound by any particular theory, the impermeable layer, e.g., a hydrophobic impermeable layer, protects the patch from saliva and other sources of moisture in order to prevent it from detaching from the application site (skin or mucosa) prematurely. In addition, the impermeable layer facilitates the application of the patch to the mucosa by providing a non-adhesive surface that will not stick the fingers.

In some embodiments, a patch according to the present disclosure provides a therapeutically effective amount of a therapeutic polypeptide for at least about 2 h, at least about 4 h, at least about 6 h, at least about 8 h, at least about 10 h, at least about 12 h, at least about 14 h, at least about 16 h, at least about 18 h, at least about 20 h, at least about 22 h, or at least about 24 h following application to a patient in need thereof. In some embodiments, the patch provides a therapeutically effective amount of a therapeutic polypeptide for at least about 4 h following application to a patient in need thereof. In some embodiments, the patch provides a therapeutically effective amount of a therapeutic polypeptide for at least about 8 h following application to a patient in need thereof. In some embodiments, the patch provides a therapeutically effective amount of a therapeutic polypeptide for at least about 12 h following application to a patient in need thereof

In some embodiments, a patch according to the present disclosure is applied to a patient in need thereof (for example, the disease site of a patient) once a day, twice a day (b.i.d.), three times per day (t.i.d.), or four times a day (q.i.d.). In some embodiments, the patch is applied to a patient in need thereof (for example, the disease site of a patient) once a day. In some embodiments, the patch is applied to a patient in need thereof (for example, the disease site of a patient) twice a day. In some embodiments, the patch is applied to a patient in need thereof (for example, the disease site of a patient) three times per day. In some embodiments, the patch is applied to a patient in need thereof (for example, the disease site of a patient) four times per day. In other embodiments, the patch is applied to a patient in need thereof on an as-needed basis.

In some embodiments, a patch according to the present disclosure is applied to a patient in need thereof (for example, the disease site of a patient) once a day for a period of about 2 to 8 weeks. In some embodiments, the patch is applied to a patient in need thereof (for example, the disease site of a patient) twice a day for a period of about 2 to 8 weeks. In some embodiments, the patch is applied to a patient in need thereof (for example, the disease site of a patient) three times per day for a period of about 2 to 8 weeks. In some embodiments, the patch is applied to a patient in need thereof (for example, the disease site of a patient) four times per day for a period of about 2 to 8 weeks. In some embodiments, the patch is applied to a patient in need thereof (for example, the disease site of a patient) once a day for a period of about 2 to 4 weeks. In some embodiments, the patch is applied to a patient in need thereof (for example, the disease site of a patient) twice a day for a period of about 2 to 4 weeks. In some embodiments, the patch is applied to a patient in need thereof (for example, the disease site of a patient) three times per day for a period of about 2 to 4 weeks. In some embodiments, the patch is applied to a patient in need thereof (for example, the disease site of a patient) four times per day for a period of about 2 to 4 weeks. In some embodiments, the patch is applied to a patient in need thereof (for example, the disease site of a patient) once a day for at least a month. In some embodiments, the patch is applied to a patient in need thereof (for example, the disease site of a patient) twice a day for at least a month. In some embodiments, the patch is applied to a patient in need thereof (for example, the disease site of a patient) three times per day for a period of at least a month. In some embodiments, the patch is applied to a patient in need thereof (for example, the disease site of a patient) four times per day for a period of at least a month. In some embodiments, the patch is applied to a patient in need thereof (for example, the disease site of a patient) once a day for at least a week. In some embodiments, the patch is applied to a patient in need thereof (for example, the disease site of a patient) twice a day for at least a week. In some embodiments, the patch is applied to a patient in need thereof (for example, the disease site of a patient) three times per day for a period of at least a week. In some embodiments, the patch is applied to a patient in need thereof (for example, the disease site of a patient) four times per day for a period of at least a week.

### Electrospun Fiber Layer

As used in the present disclosure, the term “electrospun fibers” includes fibers that are obtained by a method that involves electrostatics. The methods, referred in the art as electrohydrodynamic (EHD) methods, include electrospinning, coaxial electrospinning, coaxial electrospraying, emulsion electrospinning, and the like. Any such method can be used to prepare the fibers of the present disclosure. Accordingly, the term “electrospun fibers” is not limited to fibers obtained by electrospinning, but instead refers to fibers obtained by electrohydrodynamic methods known in the art.

In some embodiments, the electrospun fibers are uniaxial. In some embodiments, the electrospun fibers are not coaxial. Coaxial fibers are described, for example, in H. Qu, et al. J. Mater. Chem. A, 2013, 1, 11513-11528.

In some embodiments, the electrospun fibers or the fiber layer of the patches of the present disclosure comprises about 0.01% to about 50.0% (wt./wt. %), e.g., about 0.01%, about 0.1%, about 0.5%, about 1%, about 2.5%, about 5%, about 7.5%, about 10%, 12.5%, about 15%, about 17.5%, about 20%, 22.5%, about 25%, about 27.5%, about 30%, 32.5%, about 35%, about 37.5%, about 40%, 42.5%, about 45%, about 47.5%, or about 50%, of the therapeutic polypeptide, including all ranges and values therebetween. In some embodiments, the electrospun fiber or fiber layer comprises about 1.0% to about 10.0% (wt./wt. %) of the therapeutic polypeptide. In some embodiments, the electrospun fiber or fiber layer comprises about 1.0% to about 5.0% (wt./wt. %) of the therapeutic polypeptide.

In general, the electrospun fibers of the present disclosure are provided in a layer, which when applied to the skin, mucosa or a humid internal surface of the body adhere to the surface. In some embodiments, a therapeutic polypeptide (e.g., antibody, protein, peptide, and the like) can be homogeneously distributed in the electrospun fibers, whereby the concentration of drug substance per surface area of the layer is constant and a dose of the drug substance can easily be determined by using a measured area of the layer.

In some embodiments, the electrospun fibers of the present disclosure comprise one or more polymers, a therapeutically effective amount of a therapeutic polypeptide, and, optionally, an absorption enhancer, each of which is described in more detail below.

### Polymers of the Electrospun Fibers

In some embodiments, the electrospun fibers of the present disclosure comprise one or more polymers selected from the group consisting of dextran, polyethylene oxides, alginate, tragacanth, carrageenan, pectin, gelatin, guar, xanthan, gellan, fibronectin, collagen, hyaluronic acid, chitosan, cellulosic polymers such as methylcellulose, hydroxypropylmethylcellulose (HPMC), hydroxyethylcellulose, cellulose acetates, carboxymethylcellulose and alkali salts thereof, polymers of acrylic acids (PAA derivatives), chitosan, lectins, thiolated polymers, polyox WSRA, PAA-co-PEG (PEG is polyethylene glycol), polylactic acid, polyglycolic acid, poly(butylene succinate), and mixtures thereof

In some embodiments, the electrospun fibers of the present disclosure comprise one or more polymers selected from the group consisting of polyvinylpyrrolidone (PVP), acrylates and acrylic copolymers (e.g., Eudragit®), ethylcellulose (EC), hydroxypropylcellulose (HPC), polyvinyl alcohol, carboxymethylcellulose, and mixtures thereof. In some embodiments, the one or more polymers is selected from polyvinylpyrrolidone (PVP), hydroxypropylcellulose (HPC) and mixtures thereof. In some embodiments, the one or more polymers is polyvinyl alcohol or carboxymethylcellulose.

In some embodiments, the electrospun fibers of the present disclosure comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, polycaprolactone (PCL), dextran and gelatin. In some embodiments, the electrospun fibers comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the electrospun fibers of the present disclosure comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the one or more polymers comprise: (a) polyvinylpyrrolidone and an ammonio methacrylate copolymer; (b) polyvinylpyrrolidone and polyethylene glycol; (c) polyvinylpyrrolidone and polyethylene oxide; (d) polyvinylpyrrolidone and dextran; (e) polyvinylpyrrolidone and gelatin; (f) polyvinylpyrrolidone and polyvinyl alcohol; (g) polyvinylpyrrolidone, an ammonio methacrylate copolymer and polyethylene glycol; (h) polyvinylpyrrolidone, an ammonio methacrylate copolymer and polyethylene oxide; (i) polyvinylpyrrolidone, an ammonio methacrylate copolymer and dextran; (j) polyvinylpyrrolidone, an ammonio methacrylate copolymer and gelatin; or (k) polyvinylpyrrolidone, an ammonio methacrylate copolymer and polyvinyl alcohol.

In some embodiments, the one or more polymers comprise polyvinylpyrrolidone and ammonio methacrylate copolymer. In some embodiments, the weight ratio of polyvinylpyrrolidone to ammonio methacrylate copolymer in the electrospun fibers is about 0.1 to about 10, including, about 0.1, about 0.25, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to ammonio methacrylate copolymer is about 0.5 to about 5. In some embodiments, the weight ratio of polyvinylpyrrolidone to ammonio methacrylate copolymer is about 0.5 to about 2.

In some embodiments, the one or more polymers comprise polyvinylpyrrolidone and polyethylene glycol. In some embodiments, the weight ratio of polyvinylpyrrolidone to polyethylene glycol in the electrospun fibers is about 0.15 to about 10, e.g., including 0.15, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyethylene glycol is about 0.25 to about 5. In some embodiments, the weight ratio of polyvinylpyrrolidone to polyethylene glycol is about 0.5 to about 2.

In some embodiments, the one or more polymers comprise polyvinylpyrrolidone and polyethylene oxide. In some embodiments, the weight ratio of polyvinylpyrrolidone to polyethylene oxide in the electrospun fibers is about 0.3 to about 10, including, about 0.3, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyethylene oxide is about 0.3 to about 5. In some embodiments, the weight ratio of polyvinylpyrrolidone to polyethylene oxide is about 0.5 to about 2.

In some embodiments, the one or more polymers comprise polyvinylpyrrolidone and dextran. In some embodiments, the weight ratio of polyvinylpyrrolidone to dextran in the electrospun fibers is about 0.3 to about 10, including about 0.3, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to dextran is about 0.3 to about 5. In some embodiments, the weight ratio of polyvinylpyrrolidone to dextran is about 0.5 to about 2.

In some embodiments, the one or more polymers comprise polyvinylpyrrolidone and gelatin. In some embodiments, the weight ratio of polyvinylpyrrolidone to gelatin in the electrospun fibers is about 0.6 to about 10, including about 0.6, about 0.8, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to gelatin is about 0.6 to about 5. In some embodiments, the weight ratio of polyvinylpyrrolidone to gelatin is about 1 to about 3.

In some embodiments, the one or more polymers comprise polyvinylpyrrolidone and polyvinyl alcohol. In some embodiments, the weight ratio of polyvinylpyrrolidone to polyvinyl alcohol in the electrospun fibers is about 0.5 to about 10, including about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyvinyl alcohol is about 0.5 to about 5. In some embodiments, the weight ratio of polyvinylpyrrolidone to polyvinyl alcohol is about 1 to about 3.

In some embodiments, the one or more polymers comprise polyvinylpyrrolidone, an ammonio methacrylate copolymer and polyethylene glycol. In some embodiments, the weight ratio of polyvinylpyrrolidone to ammonio methacrylate copolymer to polyethylene glycol in the electrospun fibers is about 1:0.5:0.1 to about 1:2:6, including all ranges and values therebetween.

In some embodiments, the one or more polymers comprise polyvinylpyrrolidone, an ammonio methacrylate copolymer and polyethylene oxide. In some embodiments, the weight ratio of polyvinylpyrrolidone to ammonio methacrylate copolymer to polyethylene oxide in the electrospun fibers is about 1:0.5:0.1 to about 1:2:3, including all ranges and values therebetween.

In some embodiments, the one or more polymers comprise polyvinylpyrrolidone, an ammonio methacrylate copolymer and dextran. In some embodiments, the weight ratio of polyvinylpyrrolidone to ammonio methacrylate copolymer to dextran in the electrospun fibers is about 1:0.5:0.1 to about 1:2:3, including all ranges and values therebetween.

In some embodiments, the one or more polymers comprise polyvinylpyrrolidone, an ammonio methacrylate copolymer and gelatin. In some embodiments, the weight ratio of polyvinylpyrrolidone to ammonio methacrylate copolymer to gelatin in the electrospun fibers is about 1:0.5:0.1 to about 1:2:1.5, including all ranges and values therebetween.

In some embodiments, the one or more polymers comprise polyvinylpyrrolidone, an ammonio methacrylate copolymer and polyvinyl alcohol. In some embodiments, the weight ratio of polyvinylpyrrolidone to ammonio methacrylate copolymer to polyvinyl alcohol in the electrospun fibers is about 1:0.5:0.1 to about 1:2:2, including all ranges and values therebetween.

In some embodiments, the electrospun fibers of the present disclosure comprise one or more polymers (as described herein), typically including hydrophilic polymers, which provide various desirable properties, e.g., providing for rheological properties suitable for electrospinning, physical properties (e.g., strength, flexibility, etc.) to provide a patch that is sufficiently durable for handling during manufacturing and use and sufficiently flexible such that it can conform to a mucosal surface, and a suitable degree of hydrophilicity for application to mucosal surfaces. Such polymers can provide bioadhesive properties to aid in adhesion to a mucosal surface. As desired, one or more additional polymers can be added to improve bioadhesion. In particular embodiments, the electrospun fibers of the present disclosure comprise polyvinylpyrrolidone and acrylic copolymers (e.g., Eudragit® copolymers), in combination with polyethylene oxide polymers. Such electrospun fiber compositions are described, for example, in U.S. Pat. No. 10,052,291, US Patent Publication Nos. 2019/0254985, and 2019/0254986, 2019/0351662.

When polyvinylpyrrolidone is used as a polymer in the electrospun fibers, it can be used in a grade having an approximate molecular weight of from 2,500 Da to 3,000,000 Da. PVP can be purchased as Kollidon® having a variety of molecular weight ranges as shown below.

| Kollidon ® | Weight average molecular weight M_{w} |
|---|---|
| 12PF | 2,000-3,000 |
| 17PF | 7,000-11,000 |
| 25 | 28,000-34,000 |
| 30 | 44,000-54,000 |
| 90F | 1,000,000-1,500,000 |

Ethylcellulose is sold under the trademark ETHOCEL™ (Dow Chemical Company) and is available in different grades. Dependent on its ethoxyl content, ethylcellulose may have different softening point and melting point temperatures. Ethylcellulose is also produced in a number of different viscosities (see Table below).

| Product | Viscosity | Ethoxyl content % | Ethoxyl content % |
|---|---|---|---|
| viscosity | range | Standard | Medium |
| designation | mPa*s | 48.0-49.5 | 45.0-46.5 |
| 4 | 3-5.5 | ETHOCEL Std. 4 | |
| 7 | 6-8 | ETHOCEL Std. 7 | |
| 10 | 9-11 | ETHOCEL Std. 10 | |
| 14 | 12.6-15.4 | ETHOCEL Std. 14 | |
| 20 | 18.22 | ETHOCEL Std. 20 | |
| 45 | 41.49 | ETHOCEL Std. 45 | |
| 50 | 45-55 | | ETHOCEL Med. 50 |
| 70 | 63-77 | | ETHOCEL Med. 70 |
| 100 | 90-110 | ETHOCEL Std. 100 | ETHOCEL Med. 100 |
| 200 | 180-220 | ETHOCEL Std. 200 | |
| 300 | 270-330 | ETHOCEL Std. 300 | |
| 350 | 250-385 | ETHOCEL Std. 4 | |

Acrylates and acrylic acid derivatives include polymethacrylates, methacrylate copolymers, acrylic copolymers and methacrylate polymers, and include acrylates sold as EUDRAGIT as well as acrylates/octaacrylamide sold as DERMACRYL 79. Non-limiting example of such acrylates are EUDRAGIT®E 12,5 (amino methacrylate copolymer), EUDRAGIT® E100 (amino methacrylate copolymer; basic butylated methacrylate copolymer), EUDRAGIT®E PO ((amino methacrylate copolymer), EUDRAGIT®L 100-55, EUDRAGIT®L 100 (methacrylic acid—methyl methacrylate copolymer 1:1), EUDRAGIT®S 100 (methacrylic acid-methyl methacrylate copolymer 1:2), EUDRAGIT®RL 100, EUDRAGIT®RL 100 (ammonio methacrylate copolymer type A), EUDRAGIT®RL PO, EUDRAGIT®RS 100 (ammonio methacrylate copolymer type B), EUDRAGIT®RS PO. EUDRAGIT®E is a cationic polymer based on dimethylaminoethyl methacrylate and other neutral methacrylic acid esters: EUDRAGIT®L and S are methacrylic acid copolymers and are cationic copolymerization products of methacrylic acid and methyl methacrylate. EUDRAGIT®RL or RS is ammonio methacrylate copolymers synthesized from acrylic acid and methacrylic acid.

Carboxymethylcellulose is available in a broad selection of viscosity grades. In some embodiments, the viscosity of carboxymethylcellulose in the electrospun fibers ranges from 10 to 100,000 mPa*s. In some embodiments, the carboxymethylcellulose is a sodium salt.

Methylcellulose is sold under the name METHOCEL™ (Dow Chemical Company) and is available in a wide range of viscosity grades. In some embodiments, the viscosity grade of methylcellulose in the electrospun fibers is from less than about 3 to greater than about 100,000 mPA*s.

HPMC is sold under the names Methocel® and Klucel®. In some embodiments, HPMC of the electrospun fibers has an average molecular weight of from about 80,000 to about 140,000.

In some embodiments, polyvinyl alcohol has a molecular weight ranging from about 20,000 Da to about 200,000. In some embodiments, polyvinyl alcohol has a molecular weight ranging from about 100,000 Da to about 200,000.

In some embodiments, polyethylene oxide (PEO) is used in grade having an approximate molecular weight of from about 100,000 to about 7,000,000. In some embodiments, the average molecular weight of from about 700,000 to about 4,000,000. Polyethylene oxide is sold under the name POLYOX™ (Dow Chemical Company) with molecular weights ranging from about 100,000 to about 7,000,000 Da.

In some embodiments, the PEO has a molecular weight of from about 100,000 to about 4,000,000 Da. In some embodiments, the PEO has a molecular weight of from about 100,000 to about 700,000 Da. In some embodiments, the PEO has a molecular weight of from about 100,000 to about 400,000 Da. In some embodiments, the PEO has a molecular weight of about 200,000 Da. In some embodiments, the PEO has a molecular weight greater than about 2,000,000 Da. In some embodiments, the PEO has a molecular weight of from about 2,000,000 Da to about 7,000,000 Da.

In some embodiments, dextran is used in grade having a molecular weight of from about 400,000 Da to about 2,000,000 Da. In some embodiments, dextran has a molecular weight of from about 500,000 Da to about 2,000,000 Da, e.g., about 700,000 Da to about 800,000 Da or from about 1,000,000 Da to about 2,000,000 Da.

In some embodiments, the one or more polymers is present in an amount ranging from about 30% to about 99.9% by weight of the electrospun fibers, including about 30%, about 35%, about 40%, about 45%, about 50%, about 55%, about 60%, about 65%, about 70%, about 75%, about 80%, about 85%, about 90%, about 95%, or about 99.9%, including all ranges and values therebetween. In some embodiments, the one or more polymers is present in an amount ranging from about 50% to about 99.9% by weight of the electrospun fibers. In some embodiments, the one or more polymers is present in an amount ranging from about 75% to about 99.9% by weight of the electrospun fibers. In some embodiments, the one or more polymers is present in an amount ranging from about 75% to about 95% by weight of the electrospun fibers. In some embodiments, the one or more polymers is present in an amount ranging from about 75% to about 90% by weight of the electrospun fibers.

### Therapeutic Polypeptides

As disclosed herein, the electrospun fibers of the present disclosure comprise a therapeutically effective amount of a therapeutic polypeptide. In some embodiments, the therapeutic polypeptide is a monoclonal antibody, a fragment antigen-binding (Fab fragment) antibody, a single-chain variable fragment, a full antigen, a fragmented antigen, a protein, a peptide, or an enzyme.

In some embodiments, the therapeutic polypeptide is a monoclonal antibody. In some embodiments, the monoclonal antibody is selected from the group consisting of loncastuximab, balstilimab, ansuvimab, bimekizumab, omburtamab, tralokinumab, evinacumab, sutimlimab, aducanumab, teplizumab, dostarlimab, margetuximab, naxitamab, inolimomab, oportuzumab, narsoplimab, belantamab, tafasitamab, satralizumab, inebilizumab, sacituzumab, teprotumumab, isatuximab, eptinezumab, trastuzumab, enfortumab, crizanlizumab, brolucizumab, polatuzumab, risankizumab, romosozumab, caplacizumab, ravulizumab, emapalumab, cemiplimab, fremanezumab, moxetumomab, galcanezumab, lanadelumab, mogamuizumab, erenumab, tildrakizumab, ibalizumab, burosumab, durvalumab, emicizumab, benralizumab, ocrelizumab, guselkumab, inotuzumab, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alicrocumab, mepolizumab, evolocumab, dinutuximab, secukinumab, nivolumab, blinatumomab, pembrolizumab, ramucirumab, vedolizumab, siltuximab, ustekinumab, obinutuzumab, certolizumab pegol, ranibizumab, abcixumab, raxibacumab, pertuzumab, brentuximab, belimumab, ipilimumab, denosumab, tocilizumab, ofatumumab, canakinumab, golimumab, ustekinumab, catumaxomab, eculizumab, panitumumab, natalizumab, bevacizumab, cetuximab, efalizumab, omalizumab, tositumomab, ibritumomab, ibritumomab, adalimumab, alemtuzumab, gemtuzumab, infliximab, palivizumab, basilixumab, daclizumab, rituximab, edrecolomab, nebacumab, and muromonab-CD₃.

In some embodiments, the therapeutic polypeptide is a Fab fragment antibody. In some embodiments, the Fab fragment antibody is selected from the group consisting of certolizumab pegol, ranibizumab, abcixumab, abrezekimab, citatuzumab, lampalizumab, onartuzumab, tadocizumab, arcitumomab, sulesomab, and imiciromab.

In some embodiments, the therapeutic polypeptide is a single-chain variable fragment (scFv). In some embodiments, the scFV is selected from the group consisting of brolucizumab, duvortuxizumab, efungumab, flotetuzumab, ganitumab, letolizumab, oportuzumab monatox, pexelizumab, vobarilizumab.

In some embodiments, the therapeutic polypeptide is a protein. In some embodiments, the protein is selected from the group consisting of insulin, corticotropin, secretin, growth hormone GH, pegvisoman, mecasermin, factor VIII, factor IX, erythropoietin, filgrastim, oprelvekin, human follicle-stimulating hormone (FSH), human chorionic gonadotropin (HCG), palifermin, becaplermin, lepirudin, anakinra, interleukin 2 (IL2), interferons, peginterferon, etanercept, alefacept, abatacept, rilonacept, romiplostim, and belatacept.

In some embodiments, the therapeutic polypeptide is a peptide. In some embodiments, the peptide is selected from the group consisting of bradykinin, bivalirudin, buserelin, enfuvirtide, eptifibatide, glatiramer, gramicidin D, lepirudin, leuprolide, liraglutide, lucinactant, octreotide, exenatide, nesiritide, oxytocin, pramlintide, salmon calcitonin, sermorelin, teduglutide, and thymalfasin. In some embodiments, the peptide is an antimicrobial peptide. In some embodiments, the antimicrobial peptide is selected from the group consisting of bacitracin, boceprevir, dalbavancin, daptomycin, enfuvirtide, oritavancin, teicoplanin, telaprevir, telavancin, vancomysin, and guavanin 2.

In some embodiments, the therapeutic polypeptide is an enzyme. In some embodiments, the enzyme is selected from the group consisting of lysozyme, collagenase, β-glucocerebrosidase, alglucosidase-α, laronidase, idursulfase, galsulfase, agalsidase-β, lactase, pancreatic enzymes, adenosine deaminase, tissue plasminogen activator (tPA), factor VIIa, trypsin, botulinum toxin, papain, L-asparaginase, rasburicase, and streptokinase.

In some embodiments, the therapeutic polypeptide is an antigen. In some embodiments, the antigen is a full antigen. In some embodiments, the antigen is a fragment antigen. In some embodiments, the antigen is effective for treating an autoimmune disease, as described in Hirsch D, Ponda P. “Antigen-based immunotherapy for autoimmune disease: current status” Immunotargets Ther. 2015; 4:1-11, which is herein incorporated by reference in its entirety. In some embodiments, the antigen is selected from the group consisting of insulin, proinsulin, insulin peptides, GAD65, HSP60, myelin peptides (e.g., MBP85-99, MOG35-55, and PLP139-155), glatiramer acetate, dnaJP1, APL-1, P140, IPP-201101, NPL001, NPL002, and NPL003.

In some embodiments, the therapeutic polypeptide is about 0.1% to about 15.0% (wt./wt. %) of the electrospun fiber or fiber layer. In some embodiments, the therapeutic polypeptide is about 0.1% to about 10% (wt./wt. %) of the electrospun fiber or fiber layer. In some embodiments, the therapeutic polypeptide is about 0.1% to about 5.0% (wt./wt. %) of the electrospun fiber or fiber layer. In some embodiments, the therapeutic polypeptide is about 1% to about 10% (wt./wt. %) of the electrospun fiber or fiber layer. In some embodiments, the therapeutic polypeptide is about 1% to about 5.0% (wt./wt. %) of the electrospun fiber or fiber layer.

The present disclosure contemplates electrospun fibers combining any of the therapeutic polypeptides described herein with any of the aforementioned polymers.

### Absorption Enhancers

As described herein, a buccal drug delivery system has potential as a non-invasive route of drug administration, with the advantages of avoidance of the first-pass metabolism, sustained therapeutic action and better patient compliance. However, transmucosal delivery of drugs by means of the buccal route can be challenging.

Accordingly, in some embodiments, the electrospun fibers of the present disclosure further comprise an absorption enhancer. In some embodiments, the presence of an absorption enhancer improves the absorption or permeation of a therapeutic polypeptide through mucosa, e.g., oral mucosa. In some embodiments, the presence of an absorption enhancer improves sublingual absorption or permeation of a therapeutic polypeptide. In some embodiments, the presence of an absorption enhancer improves oromucosal absorption or permeation of a therapeutic polypeptide.

In some embodiments, the absorption enhancer is selected from the group consisting of a fatty acid, a non-ionic surfactant, a polycation, a thiolated polymer, a cyclodextrin, and a cell-penetrating peptide.

In some embodiments, the absorption enhancer is a fatty acid. In some embodiments, the fatty acid is selected from the group consisting of caproic acid, caprylic acid, capric acid, sodium caprate, lauric acid, stearic acid, oleic acid, linoleic acid, and linolenic acid.

In some embodiments, the absorption enhancer is a non-ionic surfactant. In some embodiments, the non-ionic surfactant is selected from the group consisting of polysorbate (e.g., polysorbate 20, polysorbate 40, polysorbate 60, and polysorbate 80), polyethylene glycol alkyl ether (e.g., polyethylene glycol dodecyl ether, polyethylene glycol hexadecyl ether, polyethylene glycol octadecyl ether and polyethylene glycol oleyl ether), polyoxyethylene alkyl ethers, (polyoxyethylene lauryl ether, polyoxyethylene oleyl ether, and polyoxyethylene stearyl ether), Nonylphenoxypolyoxyethylene (NPPOE), laurate sucrose ester (SE), and sodium glycocholate.

In some embodiments, the absorption enhancer is a polycation. In some embodiments, the polycation is selected from the group consisting of a chitosan and its quaternary ammonium derivatives, a poly-L-arginine, an aminated gelatin, and cetylpyridinium chloride.

In some embodiments, the absorption enhancer is a thiolated polymer. In some embodiments, the thiolated polymer is selected from the group consisting of carboxymethyl cellulose-cysteine, polycarbophil (PCP)-cysteine, chitosan-thiobutylamidine, chitosan-thioglycolic acid, chitosan-thioethylamidine, chitosan iminothiolane, chitosan-glutathione conjugates, polyacrylic acid-cysteine, polymethacrylic acid-cysteine, and hyaluronicacid-L-cysteine.

In some embodiments, the absorption enhancer is a cyclodextrin. In some embodiments, the cyclodextrin is selected from the group consisting of a methylated β-cyclodextrin,hy droxypropyl-β-cyclodextrin, and sulphobutylether-β-cyclodextrin.

In some embodiments, the absorption enhancer is a cell-penetrating peptide. In some embodiments, the cell-penetrating peptide is selected from the group consisting of penetratin, Tat (YGRKKKRRQRRR), R6, R8, R9, pVEC (LLIILRRRIRKQAHAHSK), RRL helix (RRLRRLLRRLRRLLRRLR), shuffle (RWFKIQMQIRRWKNKK), and penetramax (KWFKIQMQIRRWKNKR).

In some embodiments, the patches of the present disclosure deliver an effective amount of a therapeutic polypeptide transdermally. Like transbuccal drug delivery, transdermal delivery has several advantages including the ability to achieve steady-state drug levels, bypass the hepatic first-pass metabolism, increase patient compliance and reduce gastrointestinal (GI) adverse effects.

In some embodiments, the efficiency of transdermal delivery is improved by addition of an absorption enhancer (i.e., a penetration enhancer) described herein. In some embodiments, the absorption enhancer is a fatty acid (e.g., oleic acid), terpene, surfactant, propylene glycol, pyrrolidone derivative (e.g., N-methyl-2-pyrroldone), carbamic acid derivative, dioxolone derivative, and dioxane derivative. In some embodiments, the absorption enhancer is a cell-penetrating peptide (e.g., penetratin), a skin-penetrating peptide (hyaluronic acid conjugated to phospholipid), or antimicrobial peptide (e.g., magainin, GIGKFLHSAKKFGKAFVGEIMNS). In some embodiments, the absorption enhancer comprises a nanocarrier, including, but not limited to liposomes, niosomes, transfersomes, and ethosomes. Absorption enhancers relevant to the present disclosure include those described in B. Chaulagain et al. “Passive Delivery of Protein Drugs Through Transdermal Route” Artificial Cells, Nanomedicine, and Biotechnology 2018, 46, 5471-5487, incorporated herein by reference in its entirety.

In some embodiments, the efficiency of transdermal delivery is improved by the application of heat to the skin, as described in S. Szunerits et al. “Heat: A Highly Efficient Skin Enhancer for Transdermal Drug Delivery” Front. Bioeng. Biotechnol. 2018, 6(15), 1-13, incorporated herein by reference in its entirety. For example, in some embodiments, heat-assisted microporation is used to create transport channels in the skin for improved permeation of therapeutic polypeptides. In some embodiments, thermal ablation with lasers, based on the selective removal of the stratum corneum (SC) by localized microsecond heat pulses, is used to increase permeability of the skin's outer barrier to therapeutic polypeptides.

### Therapeutically Active Agents

In some embodiments, the electrospun fibers of the therapeutic polypeptide-containing patches of the present disclosure further comprise a therapeutically effective amount of an additional therapeutic agent.

In some embodiments, the additional therapeutic agent is a steroid, an analgesic, a calcineurin inhibitor, a barbiturate, a benzodiazepine, or a mixture thereof. In some embodiments, the additional therapeutic agent is a steroid. In some embodiments, the additional therapeutic agent is an analgesic. In some embodiments, the additional therapeutic agent is a calcineurin inhibitor. In some embodiments, the additional therapeutic agent is a barbiturate. In some embodiments, the additional therapeutic agent is a benzodiazepine.

In some embodiments, the additional therapeutic agent is about 0.01% to about 10.0% (wt./wt. %) of the electrospun fiber layer, including about 0.01%, about 0.02%, about 0.03%, about 0.04%, about 0.05%, about 0.06%, about 0.07%, about 0.08%, about 0.09%,about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%, including all ranges and values therebetween. In some embodiments, the additional therapeutic agent is about 0.05% to about 5.0% (wt./wt. %) of the electrospun fiber layer.

In some embodiments, the electrospun fibers of the present disclosure further comprise a steroid. In some embodiments, the steroid is selected from the group consisting of amcinonide, betamethasone, budesonide, clobetasol, clobetasone, cortisone, desonide, desoxycortisone, desoximethasone, dexamethasone, diflucortolon, diflorasone, flucortisone, flumethasone, flunisolide, fluocinonide, fluocinolon, fluorometholone, fluprednisolone, flurandrenolide, fluticasone, halcinonide, halobetasol, hydrocortisone, meprednisone, methylprednisone, mometasone, paramethasone, prednicarbate, prednisone, prednisolone and triamcinolone or a pharmaceutically acceptable ester or acetonide thereof. In some embodiments, the steroid is selected from the group consisting of betamethasone, budesonide, clobetasol, clobetasone, desoximethasone, diflucortolon, diflorasone, fluocinonide, fluocinolon, halcinonide, halobetasol, hydrocortisone, mometasone and triamcinolone or a pharmaceutically acceptable ester thereof. In some embodiments, the steroid is selected from the group consisting of clobetasol propionate, mometasone, fluocinonide, betamethasone, and dexamethasone. In some embodiments, the steroid is clobetasol propionate. In some embodiments, the steroid is mometasone.

In some embodiments, the electrospun fibers of the present disclosure comprise an analgesic selected from the group consisting of local anesthetics (e.g., lidocaine, benzocaine, and the like), acetaminophen, nonsteroidal anti-inflammatory drugs (NSAIDs), benzamidine hydrochloride, etomidate, ketamine, propofol, gabapentin, tramadol, and pregabalin. In some embodiments, the analgesic is selected from the group consisting of lidocaine, acetaminophen, nonsteroidal anti-inflammatory drugs (NSAIDs, e.g., aspirin, naproxen, ibuprofen, diclofenac, etc.), etomidate, ketamine, propofol, gabapentin, tramadol, and pregabalin. In some embodiments, the NSAID is selected from the group consisting of diclofenac, diflunisal, etodolac, ibuprofen, indomethacin, ketoprofen, ketorolac, nabumetone, naproxen, oxaprozin, piroxicam, salsalate, sulindac, and tolmetin. In some embodiments, the NSAID is selected from the group consisting of aspirin, naproxen, ibuprofen, and diclofenac. In some embodiments, the analgesic is a topical analgesic. In some embodiments, the analgesic is selected from the group consisting of lidocaine, benzocaine, prilocaine, xylocaine, tetracaine, capsaicin, menthol, and methyl salicylate. In some embodiments, the analgesic is lidocaine or articaine. In some embodiments, the analgesic is lidocaine.

In some embodiments, the electrospun fibers of the present disclosure comprise a calcineurin inhibitor selected from the group consisting of tacrolimus, picrolimus, pimecrolimus, cyclosporine, and voclosporin. In some embodiments, the calcineurin inhibitor is selected from the group consisting of tacrolimus, picrolimus, and cyclosporine.

In some embodiments, the electrospun fibers of the present disclosure comprise a barbiturate selected from the group consisting of secobarbital, butabarbital, methobarbital, pentobarbital, phenobarbital, aprobarbital, primidone, amobarbital, methohexital, thiamylal, and thiopental. In some embodiments, the barbiturate is selected from the group consisting of amobarbital, methohexital, thiamylal, and thiopental.

In some embodiments, the electrospun fibers of the present disclosure comprise a benzodiazepine selected from the group consisting of alprazolam, bentazepam, bromazepam, brotizolam, camazepam, chlordiazepoxide, clobazam, clonazepam, clonazolam, clorazepate, clotiazepam, diazepam, flumazenil, flunitrazepam, flurazepam, halazepam, loprazolam, lorazepam, medazepam, mexazolam, midazolam, oxazepam, prazepam, quazepam, temazepam, triazolam, zaleplon, and zolpidem. In some embodiments, the benzodiazepine is selected from the group consisting of diazepam, lorazepam, and midazolam.

### Impermeable Layer

To prevent premature detachment from the skin or mucosal surface as well as other therapeutic benefits, the patches of the present disclosure comprise an impermeable layer.

In some embodiments, the impermeable layer is provided as a coating disposed on a drug-containing layer. In some embodiments, the impermeable layer is co-spun with the drug-containing, and is therefore integrated into the electrospun fibers.

In some embodiments, the impermeable layer is a backing layer. In some embodiments, the impermeable layer is a hydrophobic impermeable layer.

In some embodiments, the impermeable layer protects the drug-containing layer(s) from moisture or saliva. In some embodiments, the impermeable layer protects the drug-layer from being washed away from the application site, which results in the desired local therapeutic effect being reduced or eliminated. In some embodiments, the impermeable layer functions as an occlusive layer, which drives the penetration of drug substance into the skin or mucosa.

In some embodiments, the impermeable layer comprises one or more biodegradable polyesters. In some embodiments, the biodegradable polyester is an aliphatic polyester. In some embodiments, the biodegradable polyester is an aromatic polyester. In some embodiments, the impermeable layer comprises one or more polymers selected from the group consisting of poly(caprolactone), poly(glycolic acid), poly(lactic acid), poly(lactide-co-glycolide), poly(butylene succinate), poly(butylene succinate-co-adipate), poly(p-dioxanone), and poly(trimethylene carbonate). In some embodiments, the impermeable layer comprises one or more polymers selected from the group consisting of poly(caprolactone), poly(glycolic acid), poly(lactic acid), and poly(lactide-co-glycolide). In some embodiments, the impermeable layer comprises poly (caprol actone).

In some embodiments, the impermeable layer comprises polyethylene-co-vinyl acetate, ethylcellulose, poly(caprolactone), carbothane or polysoftane. In some embodiments, the impermeable layer comprises acrylates/octylacrylamide copolymer (sold under the name DERMACRL® 79), amino methacrylate copolymer (EUDRAGIT®), dimethylaminoethyl methacrylate, methacrylate, methyl methacrylate (e.g. EUDRAGIT ®E 100), or other acrylates.

In some embodiments, the impermeable layer comprises about 5-70% by weight of the patch, including about 5%, about 10%, about 15%, about 20%, about 25%, about 30%, about 35%, about 40%, about 45%, about 50%, about 55%, about 60%, about 65%, or about 70%, including all values and ranges therebetween, by weight of the patch. In some embodiments, the impermeable layer comprises about 5-50% by weight of the patch. In some embodiments, the impermeable layer comprises about 10-30% by weight of the patch.

### Methods of Treatment

In some embodiments, the present disclosure provides methods of treating or preventing a disease or disorder in a patient in need thereof by administering a polypeptide-containing patch as disclosed herein. In some embodiments, the patch comprises an electrospun fiber layer, wherein the electrospun fibers comprise: one or more polymers of the present disclosure and a therapeutically effective amount of a therapeutic polypeptide of the present disclosure; and (b) an impermeable layer.

In some embodiments, a polypeptide-containing patch of the present disclosure is administered (i.e., applied) to the mucosa of a patient. In some embodiments, the mucosa is the oral mucosa of a patient (i.e., transbuccal or transmucosal administration). In some embodiments, administering the patch to the oral mucosa of a patient provides systemic delivery of a therapeutic polypeptide.

In some embodiments, a patch of the present disclosure is administered (i.e., applied) to the skin of a patient (i.e. transdermal administration). In some embodiments, administering the patch to the skin of a patient provides systemic delivery of a therapeutic polypeptide.

The patches of the present disclosure may be administered by, for example, placing the patch on a mucosal surface in need of treatment with the polypeptide-containing electrospun fiber layer contacting the mucosal surface. The patch may be applied by hand or using an applicator.

In some embodiments, the present disclosure provides a method of treating a microbial infection in a patient in need thereof. In some embodiments, the microbial infection is a systemic microbial infection. In some embodiments, the microbial infection is a bacterial, viral, or fungal infection. In some embodiments, the microbial infection is a bacterial infection. In some embodiments, the bacterial infection is a drug-resistant bacterial infection. In some embodiments, the microbial infection being treated by a patch of the present disclosure is an oral infection.

In some embodiments, the present disclosure provides a method of treating cancer, an inflammatory condition, or an autoimmune disease.

In some embodiments, the present disclosure provides a method of treating a condition in a patient in need thereof, the method comprising administering a patch of disclosed herein, wherein the condition is selected from the group consisting of recurrent aphthous stomatitis, oral lichen planus, pemphigoid, pemphigus, oral mucositis, Graft versus host disease (GvHD), Bechet's disease, lupus erythematosus, vulva lichen planus, Lipschutz ulcers, lichen simplex chronicus, vulva psoriasis, allergies, autoimmune disorders or disorders with an immune component (multiple sclerosis, rheumatoid arthritis), osteoporosis, inflammatory bowel disease, hypercholesterolemia, asthma, hematologic malignancies, solid tumors, allograft rejection, and infectious organisms.

In some embodiments, the present disclosure provides a method of treating OLP in a patient in need thereof, the method comprising administering a polypeptide-containing patch disclosed herein. In some embodiments, the polypeptide-containing patch comprises an anti-TNF-α antibody. In some embodiments, the anti-TNF-α antibody is selected from the group consisting of infliximab, certolizumab, golimumab, and adalimumab. In some embodiments, the anti-TNF-α antibody comprises IgG F(ab) fragments.

In some embodiments, a patch of the present disclosure useful for treating recurrent aphthous stomatitis, oral lichen planus, pemphigoid, pemphigus, Graft versus host disease (GvHD), Behcet's disease, lupus erythematosus, vulva lichen planus, Lipschutz ulcers, lichen simplex chronicus, vulva psoriasis, and cancer comprises a steroid (e.g., clobetasol propionate or mometasone) described herein as an active agent.

In some embodiments, a patch of the present disclosure useful for treating aphthous stomatitis, Behcet's disease, oral mucositis, Lipschutz ulcers comprises an analgesic described herein (e.g., lidocaine) as an active agent.

In some embodiments, a patch of the present disclosure useful for treating GvHD comprises a calcineurin inhibitor described herein (e.g., tacrolimus or cyclosporine) as an active agent.

In some embodiments, a patch of the present disclosure useful for treating lupus erythematosus comprises an analgesic described herein (e.g., an NSAID such as naproxen or ibuprofen) as an active agent.

In some embodiments, a patch of the present disclosure is administered once a day. In some embodiments, the patch is administered twice a day. In some embodiments, the patch is administered three times a day. In some embodiments, the patch is administered four times a day. In some embodiments, the patch is administered once a day for a period of about 1-8 weeks. In some embodiments, the patch is administered twice a day for a period of about 1-8 weeks. In some embodiments, the patch is administered three times a day for a period of about 1-8 weeks. In some embodiments, the patch is administered four times a day for a period of about 1-8 weeks. In some embodiments, the patch is administered once a day for a period of about 2-4 weeks. In some embodiments, the patch is administered twice a day for a period of about 2-4 weeks. In some embodiments, the patch is administered three times a day for a period of 2-4 weeks. In some embodiments, the patch is administered four times a day for a period of 2-4 weeks. In some embodiments, the patch is administered once a day for at least a month. In some embodiments, the patch is administered twice a day for at least a month. In some embodiments, the patch is administered three times a day for at least a month. In some embodiments, the patch is administered four times a day for at least a month. In some embodiments, the patch is administered once a day for at least a week. In some embodiments, the patch is administered twice a day for at least a week. In some embodiments, the patch is administered three times a day for at least a week. In some embodiments, the patch is administered four times a day for at least a week. In some embodiments, the patch is administered to the patient as needed.

### Method of Preparation

In one aspect, the present disclosure provides a method of preparing a patch as described herein, the process comprising (a) combining a solvent with one or more polymers and a therapeutic polypeptide of the present disclosure to provide an electrospinning mixture; (b) electrospinning the electrospinning mixture of step (a) to provide electrospun fibers; and (c) attaching an impermeable backing layer to the electrospun fibers of step (b).

In some embodiments, the conductivity of the electrospinning mixture of step (a) is from about 150 μS/cm to about 600 μS/cm, e.g., about 150 μS/cm, 175 μS/cm, 200 μS/cm, about 225 μS/cm, about 250 μS/cm, about 275 μS/cm, about 300 μS/cm, about 325 μS/cm, about 350 μS/cm, about 375 μS/cm, about 400 μS/cm, about 425 μS/cm, about 450 μS/cm, about 475 μS/cm, about 500 μS/cm, about 525 μS/cm, about 550 μS/cm, about 575 μS/cm, or about 600 μS/cm, including all ranges and values therebetween. In some embodiments, the conductivity of the electrospinning mixture of step (a) is from about 150μS/cm to about 300 μS/cm.

In some embodiments, the solvent of Step (a) is an aqueous alcoholic solvent. In some embodiments, the aqueous alcohol solvent comprises ethanol and water. In some embodiments, the aqueous alcohol solvent comprises about 20% to about 97% (vol./vol. %) of ethanol. In some embodiments, the aqueous alcohol solvent comprises about 40% to about 97% (vol./vol. %) of ethanol. In some embodiments, the aqueous alcohol solvent comprises about 80% to about 97% (vol./vol. %) of ethanol. In some embodiments, the aqueous alcohol solvent comprises about 50% to about 80% (vol./vol. %) of ethanol. In some embodiments, the aqueous alcohol solvent comprises about 50% (vol./vol. %) of ethanol. In some embodiments, the aqueous alcohol solvent comprises about 80% (vol./vol. %) of ethanol. In some embodiments, the aqueous alcohol solvent comprises about 97% (vol./vol. %) of ethanol.

In some embodiments, the aqueous alcoholic solvent comprises a mixture of ethanol and 2% acetic acid in phosphate-buffered saline. In some embodiments, the aqueous solvent comprises about 40% to about 80% ethanol and 20% of 2% acetic acid in phosphate-buffered saline (PBS) as the remainder. In some embodiments, the aqueous solvent comprises about 80% ethanol and about 20% of 2% acetic acid in phosphate-buffered saline (PBS).

In some embodiments, the solvent of Step (a) comprises a mixture of an alcohol and dichloromethane or chloroform. In some embodiments, the alcohol is ethanol, n-propanol, or n-butanol. In some embodiments, the alcohol is n-butanol. In some embodiments, the solvent of Step (a) is a mixture of n-butanol and chloroform. In some embodiments, the alkyl chloride is chloroform. In some embodiments, the solvent of Step (a) comprises a mixture of two solvents selected from the group consisting of dichloromethane, ethanol, isopropanol, and butanone. In some embodiments, the solvent of Step (a) comprises n-butanol and chloroform.

In some embodiments, the electrospun fibers comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the one or more polymers comprise: (a) polyvinylpyrrolidone and an ammonio methacrylate copolymer; (b) polyvinylpyrrolidone and polyethylene glycol; (c) polyvinylpyrrolidone and polyethylene oxide; (d) polyvinylpyrrolidone and dextran; (e) polyvinylpyrrolidone and gelatin; (f) polyvinylpyrrolidone and polyvinyl alcohol; (g) polyvinylpyrrolidone, an ammonio methacrylate copolymer and polyethylene glycol; (h) polyvinylpyrrolidone, an ammonio methacrylate copolymer and polyethylene oxide; (i) polyvinylpyrrolidone, an ammonio methacrylate copolymer and dextran; (j) polyvinylpyrrolidone, an ammonio methacrylate copolymer and gelatin; or (k) polyvinylpyrrolidone, an ammonio methacrylate copolymer and polyvinyl alcohol.

In some embodiments, the electrospinning mixture of Step (a) further comprises an absorption enhancer, as disclosed herein.

### EXAMPLES

### Example 1

### Preparation of Polymer Solutions and Fabrication of Bioadhesive Fibers

All electrospinning solutions contained 0.1025±0.00025 g/mL PVP and 0.1225±0.0005 g/mL Eudragit RS100 (by total solvent volume before mixing). The amounts of PVP and RS100 were added to ethanol or an ethanol/water mixture and mixed at room temperature using a magnetic stirrer until dissolved. Lysozyme was dissolved in ice cold PBS (75 mg/mL), added to the polymer solution and stirred until uniformly distributed, contributing 3 v/v % to the final solvent composition. Electrospinning was started within 1 minute of adding the lysozyme. Placebo solutions and fibers were prepared using 3 v/v % distilled water instead of lysozyme in PBS.

Electrospun fibers were fabricated using a system composed of a PHD2000 syringe pump (Havard Apparatus, Cambridge, UK) and an Alpha IV Brandenburg power source (Brandenburg, Worthing, UK). Plastic syringes (1 mL volume; Henke Sass Wolf, Tuttlingen, Germany) were used to drive the solutions into a 20-gauge blunt metallic needle (Fisnar Europe, Glasgow, UK). Electrospinning was performed at room temperature with a potential difference of 19 kV, a flow rate of 2 mL/h, and a flight path of 14 cm.

### Example 2

### Determining Physical Properties of Solutions

Conductivity and viscosity measurements were used to determine how the addition of a protein affects solution properties.

Testing was carried out on electrospinning solutions of Example 1 comprising 1% lysozyme by dry mass with different mixtures of ethanol and water as solvents and placebo solutions in 97 v/v % ethanol without lysozyme (P).

Conductivity of Polymer Solutions

Electrical conductivity of the polymeric solutions was measured using a Mettler Toledo FG3 conductivity meter (Mettler Toledo, Schwerzenbach, Switzerland), applying a conductivity standard of 1413 μS cm⁻¹ (Mettler Toledo).

Rheology of Polymer Solutions

The viscosity of the polymeric solutions was measured using an MCR 301 rheometer (Anton Paar, Graz, Austria) with a cone-plate measuring system CP25-4/IMG1 (25 mm diameter, 4° cone angle, and 253 μm truncation) at a constant temperature (25±0.1° C.) and a sample volume of approximately 0.4 mL. Logarithmic shear rate sweep tests were performed with 31 points in the range of 0.1 to 100 s -1 lasting 20 s per point and the average viscosity calculated.

Results

The morphology of electrospun fibers is influenced by processing parameters and solution properties.

Including lysozyme in PBS caused an increase in solution conductivity from 118.4±7.2 to 168.6±8.6 μS/cm (p=0.0247) and an increase in solution viscosity from 1.14±0.17 to 1.492±0.076 Pa·s (p=0.0109) in comparison to the equivalent solution prepared with distilled water. The increase in conductivity on addition of lysozyme, a cationic protein, was expected due to increased electrolyte concentration. The increase in viscosity is typical of a protein solution.

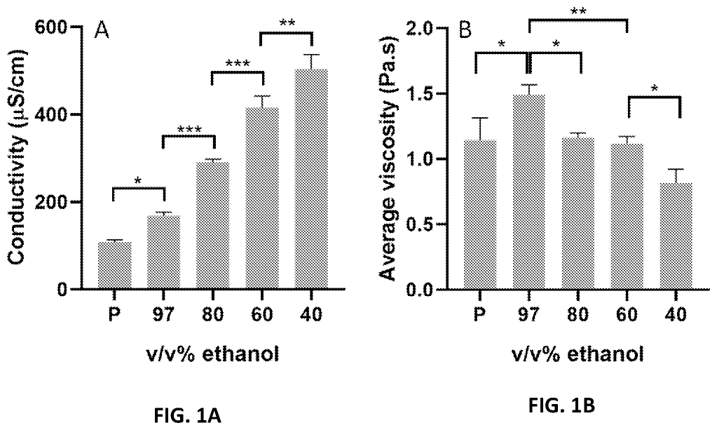

An organic solvent such as ethanol is useful for the dissolution of RS100, however ethanol can act as a protein denaturant. It was therefore hypothesized that reducing the proportion of ethanol in the electrospinning solvent mixture would improve protein activity. Accordingly, solvent mixtures with different proportions of ethanol were investigated. Decreasing the proportion of ethanol in the solution and replacing with water caused a linear increase in conductivity (R²=0.9878) reaching 504=33 μS/cm at 40 v/v % ethanol (FIG. 1A). This follows a similar trend to that previously reported for pure ethanol-water mixtures and is expected due to ethanol having a much lower conductivity than water. Reducing the proportion of ethanol from 97 to 80 v/v % reduced viscosity from 1.492±0.076 to 1.164±0.035 Pa·s (p=0.0158, FIG. 1B), although there was no significant difference in viscosity between 80 and 60 v/v %. Reducing ethanol from 60 to 40 v/v % also caused a significant decrease in viscosity from 1.118±0.054 to 0.81±0.10 Pa·s (p=0.0265, FIG. 1B). The decrease in viscosity at lower ethanol concentrations is consistent with polymers adopting a globule conformation with a smaller hydrodynamic volume due to poorer solvation. PVP in ethanol-water mixtures has previously been shown to have a reduced hydrodynamic radius at lower ethanol concentrations, suggesting a transition from chains to less solvated globules. RS100 is a less hydrophilic, water-insoluble polymer, therefore it is also likely to be poorly solvated at lower ethanol concentrations. All polymer solutions behaved approximately as Newtonian fluids with no clear shear thickening or thinning trend.

### Example 3

### Morphology of Electrospun Fibers

Lysozyme-Containing fibers

Electrospun fibers of Example 1 containing 1 w/v % lysozyme made using 97, 80, 60, and 40 v/v % ethanol mixed with distilled water as solvent, as well as placebo fibers manufactured with 97 v/v % ethanol without lysozyme were analyzed by scanning electron microscopy (SEM).

Procedure

Electrospun fibers were imaged using a TESCAN Vega3 scanning electron microscope (SEM; Tescan, Cambridge, UK). Samples were sputter coated with gold and imaged using an emission current of 10 kV. All images were processed using ImageJ software tools.' Fiber diameters were measured by ImageJ, using randomly generated coordinates and a superimposed grid to select fibers to measure. Three images were analyzed for each composition with at least 10 measurements per image.

Results

SEM of Lysozyme-Containing fibers

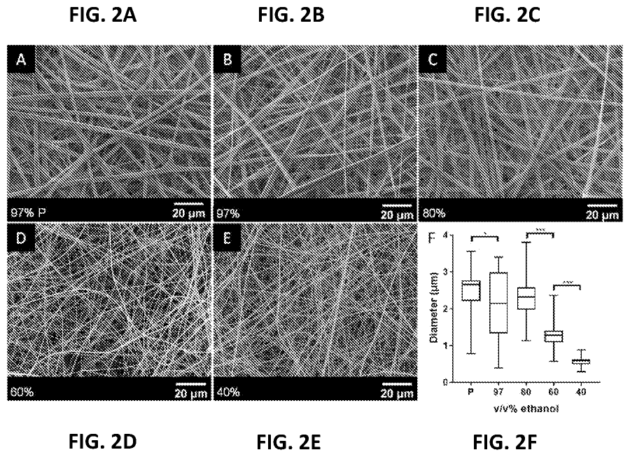

As shown in FIGS. 2A-2E, all samples had some merged fibers and almost no bead defects. No other defects were observed. Including lysozyme caused a decrease in diameter from 2.50±0.71 to 2.04±0.92 μm (FIG. 2F, p=0.0402). The reduced fiber diameter is consistent with the observed increase in conductivity, which allows the polymer solution to hold more charge, resulting in more efficient elongation in the electric field. There was no significant difference in diameter between 97 v/v % and 80 v/v % ethanol, however there was a significant decrease in fiber diameter from 2.34±0.63 μm to 1.28±0.41 μm (p<0.0001) and 0.58±0.13 μm (p<0.0001) when the ethanol content was reduced from 80 v/v % to 60 and 40 v/v % ethanol respectively. Higher solution conductivity and lower viscosity are understood to be associated with narrower fibers, therefore these measurements broadly follow the trend expected from the solutions properties.

SEM of Bradykinin-Containing Fibers

Electrospun fibers containing 0.1 v/v % bradykinin (BK) made using 100 v/v % ethanol mixed with distilled water as solvent, as well as placebo fibers manufactured with 97 v/v % ethanol without BK were analyzed by scanning electron microscopy (SEM).

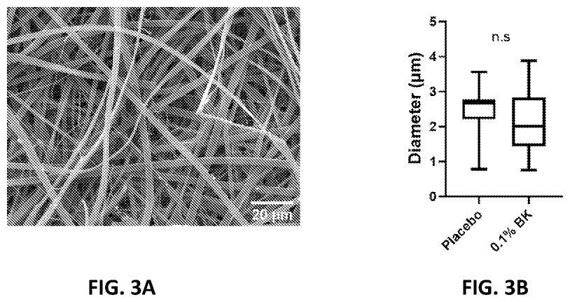

As shown in FIG. 3A, the electrospun fibers had some merged fibers and almost no bead defects. No other defects were observed. Including bradykinin caused a decrease in the median fiber diameter and an increase in the variability of fiber diameter. However, no statistically significant differences were observed between the fiber diameters of both compositions. (FIG. 3B).

SEM of Insulin-Containing Fibers

Electrospun fibers containing 1 v/v %, 3 v/v %, and 5 v/v % insulin made using 80 v/v % ethanol mixed with distilled water as solvent, as well as placebo fibers manufactured with 97 v/v % ethanol were analyzed by scanning electron microscopy (SEM).

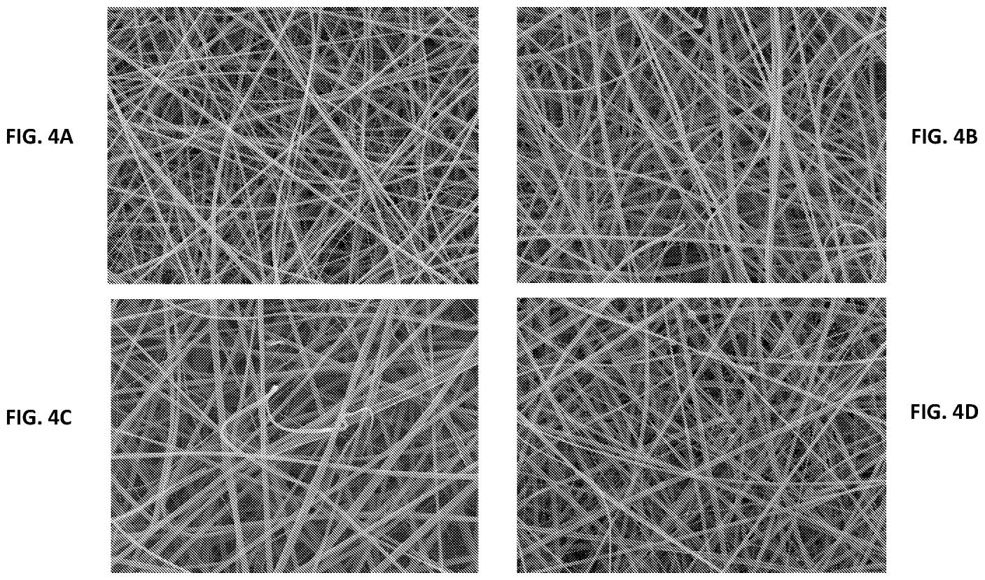

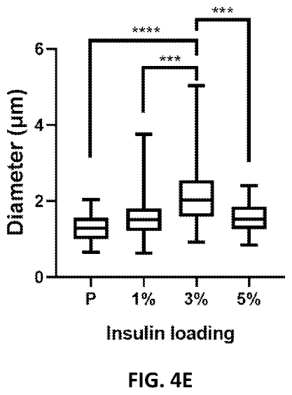

The images in FIGS. 4A-4D show that the morphology of the fibers does not change significantly as the content of insulin increases, although small circular regions appeared in the fibers comprising 5% w/w insulin (FIG. 4D), which may be regions containing a greater concentration of insulin. In general, the electrospun fibers had some merged fibers and almost no bead defects. No other defects were observed. Generally, the inclusion of insulin resulted in an increase in fiber diameter, with the inclusion of 3% w/w insulin showing the greatest effect when compared with placebo patches without insulin (PP) (FIG. 4E, p<0.0001). Interestingly, fiber diameter decreased when the insulin content was increased from 3% w/w to 5% w/w (p<0.001).

### Example 4

### Swelling and Integrity of Disclosed Electrospun Fibers

To be suitable as a mucoadhesive drug delivery system, the protein-containing fibers of the present disclosure should swell to promote adhesion, but also remain intact in a wet environment.

To evaluate these properties, lysozyme-containing electrospun fibers using different ethanol concentrations were evaluated for their degree of swelling. Specifically, pre-weighed 15 mm discs with the electrospun fibers (5-14 mg) were placed in sample tubes with 1 mL distilled water for 2 h. If intact, samples were removed, and each side pressed against a glass surface to remove excess water before reweighing. The degree of swelling was calculated as the percentage increase in mass after swelling

Results

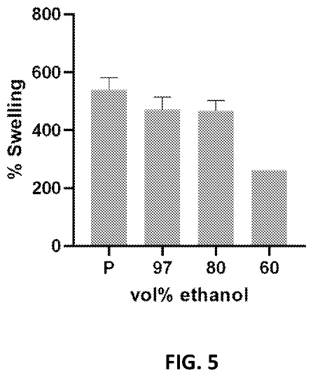

As shown in FIG. 5, all samples electrospun using 40 v/v % ethanol and one of three fibers using 60 v/v % ethanol rapidly disintegrated into small insoluble particles when added to water. In contrast, fibers electrospun from 97 and 80 v/v % ethanol and placebo fibers remained intact after 2 h and did not differ significantly in degree of swelling (p=0.4342). These data suggest that 97 v/v % or 80 v/v % ethanol are suitable solvents for the polymer system and application. The disintegration of narrower fibers may be a result of PVP-rich domains rapidly dissolving and creating discontinuations in the fibers. This is reasonable for narrower fibers, since the smaller diameter would make it easier for discontinuations to form and the higher surface area and smaller contacts between fibers facilitates the rapid dissolution of PVP.

### Example 5

### Effect of Solvent Mixture on Encapsulation Efficiency (EE) and Activity

Procedure for Measuring EE

Electrospun fibers (9-12 mg) were prepared and immersed in 2 mL PBS for 24 h. to elute the protein. A bicinchoninic acid (BCA) protein assay was used as directed to determine encapsulation efficiencies. Absorbance was measured at 562 nm using a spectrophotometer (Tecan, Theale, UK). The apparent mass fraction determined from the protein concentrations in the samples was normalized against the dry mass fraction of lysozyme in the electrospinning solution.

Procedure for Measuring Polypeptide Activity

Lysozyme enzyme activity in samples was measured using a photometric enzyme kinetic assay as previously described (REF). Briefly, 10 μL of electrospun fiber-containing supernatant diluted in PBS (1:10, v/v) was added to a clear plastic 96-well plate along with 200 μL of lyophilized Micrococcus lysodeikticus cells (0.4 mg/mL in PBS). The change in optical density at 450 nm (OD450) was measured over time for 10 minutes. Active lysozyme concentrations of the samples were interpolated from a standard curve created using lysozyme standards (0, 20, 40, 60, 80, and 100 μg/mL) and normalized against the total protein concentration.

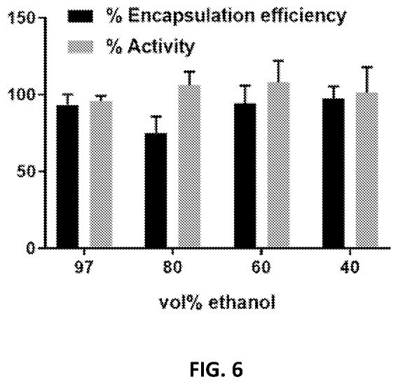

Lysozyme was eluted from the fibers and the amount released measured using the above protein assay and its activity assessed using an enzyme kinetic assay relative to freshly prepared lysozyme stock solutions. Encapsulation efficiency ranged from 75±10 to 98±8%, and no statistically significant difference between solvents was shown (FIG. 6, p=0.0750). Without being bound by any particular theory, it was initially hypothesized that reducing the concentration of ethanol in the electrospinning solution would limit protein denaturation, leading to higher enzyme activity. However, it was found that enzyme activity was close to 100% (96±3 to 108±18%) with no difference between the solvent mixtures tested. Placebo electrospun fibers were used as negative controls and showed apparent protein loading, as measured by a BCA assay, close to zero, 0.017±0.015 w/w % compared to 0.928±0.070 w/w % for lysozyme loaded fibers (p<0.005). The activity of the placebo patches was also close to zero, corresponding to an active lysozyme loading of 0.055±0.042 w/w % compared to 0.892±0.049 w/w % for lysozyme-loaded fibers (p<0.0005). This shows that the observed protein release and enzyme activity was not a false positive caused by dissolved polymer. Changing the ratio of solvents did not provide a noticeable benefit in terms of activity, therefore further experiments focused on 97 v/v % ethanol as the solvent, which gave an encapsulation efficiency of 93±7% and activity of 96±3%. The high encapsulation efficiencies reported herein are comparable to those previously reported for small molecule drugs encapsulated using uniaxial electrospinning. In addition, the polymer/solvent system described here, which has numerous advantages over prior art methods, enables encapsulation efficiencies and activity preservation superior to what has been achieved previously with uniaxial single phase and emulsion electrospinning and at least as high as that achieved with coaxial electrospinning.

### Example 6

### Homogeneity of Electrospun Fibers

To determine the distribution of PVP and lysozyme within the electrospun fibers, a novel dual fluorescent labelling approach was used. A FITC-PVP complex and Texas red conjugated lysozyme were added before electrospinning and the resulting fibers imaged using confocal microscopy.

Fluorescent Labelling

To label PVP, a complex with fluorescein isothiocyanate isomer I (FITC), as described by Aulton et al., was produced. FITC in 0.1 M pH 9.2 carbonate-bicarbonate buffer (2 mg/mL, 1mL) was added dropwise with vigorous stirring to PVP in the same buffer (25 mg/mL, 10 mL) in an opaque sample tube. The tube was sealed and incubated at room temperature for 3 h. The complex was purified by adding dropwise with stirring to acetone (500 mL) and the precipitate collected and washed with acetone (2×10 mL). Excess solvents were removed by freeze drying.

Lysozyme was labelled with Texas red sulfonyl chloride. Texas red sulfonyl chloride (1 mg) was added, with vigorous stirring until dissolved, to lysozyme in 0.1 M pH 9.2 carbonate-bicarbonate buffer (75 mg/mL, 1 mL) in an opaque sample tube. The tube was sealed and incubated at room temperature for 24 h and the product purified using gel permeation chromatography with Sephadex G-25 and PBS. The solvent was removed by freeze drying.

Results

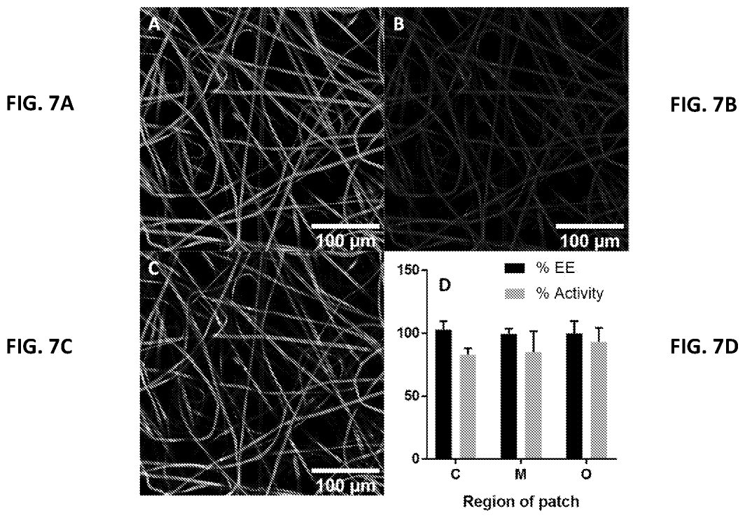

PVP appears to be homogenously distributed within the fibers, with any polymer phase separation occurring over a nanometer scale below the resolution of the microscope (FIG. 7A and FIG. 7C). Lysozyme is fully incorporated into the fibers and almost homogeneously distributed (FIG. 7B and FIG. 7C). Some aggregation is visibly present over a microscopic scale as bright regions, however this does not appear to significantly affect its activity. To determine the homogeneity of the lysozyme distribution over a macroscopic scale, the loading (% EE) and activity of different regions (center, intermediate, outer edge) of the fibers were compared (FIG. 7D). The loading and activity were similar across all regions tested indicating that the protein is homogeneously distributed at the macroscopic level.

### Example 7

Characterization of Protein Release from Electrospun Fibers

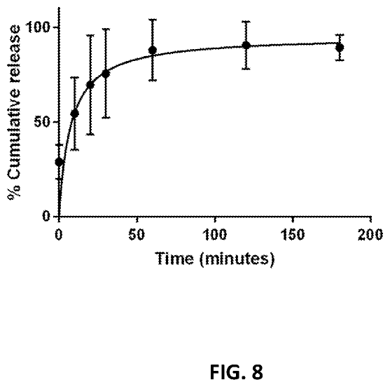

To be useful as a mucosal peptide delivery system, the electrospun fibers must release their payload over a suitable timescale. To measure the release profile, lysozyme was eluted from the electrospun fibers according to the following procedure and the active concentration measured using enzyme kinetics for up to 3 h (FIG. 8).

Procedure: Samples of electrospun membranes (20 mg) were immersed in 4 mL PBS and 10 μL samples taken at time intervals (0, 10, 20, 30, 60, 120, 180 min.) following vortexing for 5 seconds. Active lysozyme concentration was measured as previously described. To calculate cumulative release, the active concentration was normalized against the theoretical maximum concentration, assuming 100% encapsulation efficiency, release, and activity.

Lysozyme Release from Fibers

Lysozyme was rapidly released with 76±23% released within 30 minutes and 88±16% within 1 h (FIG. 8). The release then begins to plateau, reaching 90±13% after 2 h. Without being bound by any particular theory, it is believed that the release of protein is likely to be facilitated by rapid water penetration due to the dissolution of PVP and the swelling of RS100. The lack of sheath layer associated with coaxial electrospinning also increases release rate by decreasing the diffusion path required for release. Proteins previously encapsulated in water-insoluble polymer fibers have shown considerably slower release rates. Compared to other polymer systems, the delivery system reported here is unique in that it enables protein release over timescales relevant for delivery to the oral mucosa without rapidly dissolving.

Bradykinin Release from Fibers

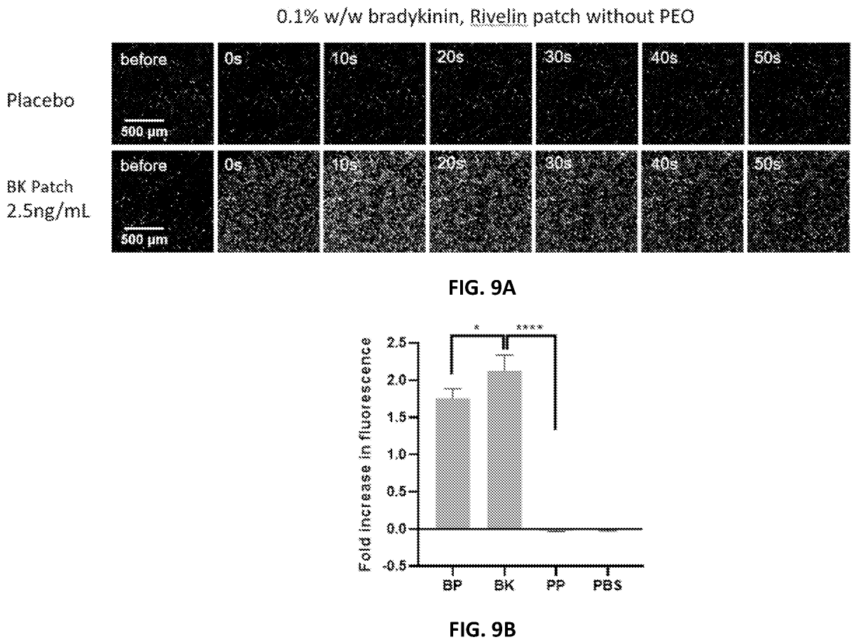

As shown in FIG. 9A, bradykinin-loaded patches were able to release their content after immersion in PBS. This was demonstrated by the detection of a significant increase in fluorescence, in comparison with placebo patches that did not contain any bradykinin. FIG. 9B shows that the quantified increase in fluorescence caused by the bradykinin-loaded patches (BP) was comparable to that caused by bradykinin dissolved in PBS (BK). No fluorescence was detected when placebo patches (PP) or pure buffer (PBS) was used.

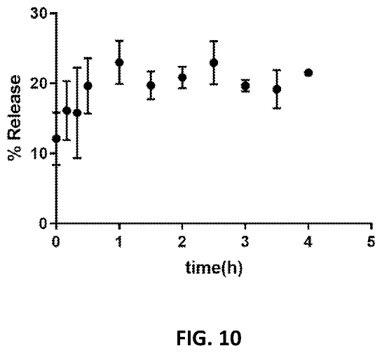

As shown in FIG. 10, about 20% of the bradykinin in the electrospun fibers was released within 30 minutes, with maximum release achieved in 1 h. Between 1 h and 4 h, release of BK plateaued.

Insulin Release from Fibers

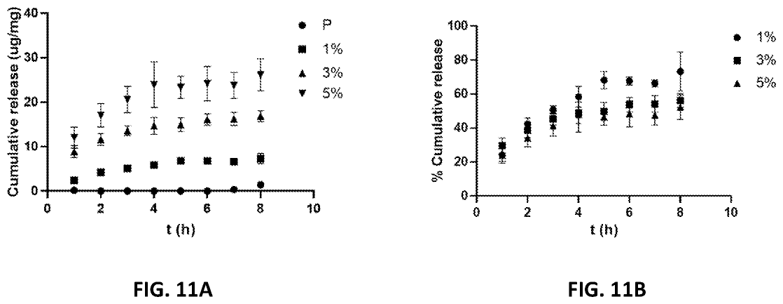

FIG. 11A shows that a concentration-dependent release of insulin occurs from the electrospun fibers. FIG. 11B shows that between 20-30% insulin was released within 1 h. For the electrospun fibers comprising 3% and 5% insulin, release plateaued after 4 h. When 1% insulin was used, a maximum release of about 70% was reached at the 5 h timepoint, followed by a plateau.

### Example 8

Fabrication of Backing Layer, Adhesion, and Effect on Activity

A film comprising hydrophobic polycaprolactone (PCL) was introduced to act as a backing layer and promote unidirectional delivery. Such a layer can also protect against mechanical forces in the mouth as previously described in dual-layer patches for the unidirectional delivery of clobetasol to the oral mucosa (see M. E. Santocildes-Romero, et al. ACS Appl. Mater. Interfaces, 2017, 9, 11557-11567, incorporated herein by reference in its entirety).

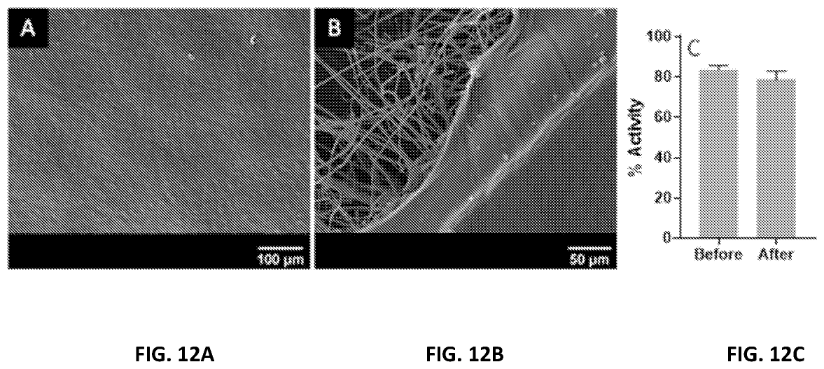

Backing layer preparation: The hydrophobic backing layer (BL) was prepared by electrospinning a PCL solution on top of the bioadhesive PVP/RS100 layer. PCL was added to a blend of DCM and DMF (90:10 v/v %) and stirred at room temperature until dissolved to prepare a solution of concentration 10 w/v %. To enhance the attachment between the bioadhesive and backing layer a thermal treatment was applied by heating at 65° C. for 15 minutes in a dry oven. PCL was electrospun on top of the protein-loaded mucoadhesive layer of Example 1 and the dual-layer patch heated at 65° C. to melt the PCL fibers into a continuous film. SEM micrographs show the fabrication of patches with a continuous backing film and that the fibrous structure of the lower layer was preserved (FIG. 12A and FIG. 12B).

Adhesion Study

The adhesive properties of electrospun fibers with backing layers were investigated in vitro. The objective was to assess whether lysozyme had a detrimental effect on patch adhesion or structural integrity.

Procedure: For each composition, two discs of 1 cm diameter were cut from three sets of electrospun fibers (n=6) and applied to 20 μL droplets of PBS on a plastic petri dish with gentle pressure from the index finger for 5 s. The petri dishes were filled with PBS (20 mL) and then incubated on an orbital shaker at 250 rpm at room temperature for a total of 2 weeks. The samples were inspected daily to observe any detachment of the backing layer.

Results: Both the lysozyme-containing patches and the placebo patches remained attached until the end of the experiment, suggesting that lysozyme does not disrupt membrane integrity, adhesion, or attachment of the backing layer.

Evaluation of Enzyme Activity

To calculate cumulative release, the active concentration was normalized against the theoretical maximum concentration, assuming 100% encapsulation efficiency, release, and activity.

Activity measurements revealed that thermal treatment did not cause significant loss of enzyme activity (FIG. 12C, p=0.8326). Applying dry heat at 100° C. for 1 week resulted in a decrease in activity from 84±4% to 30±16% (p=0.0017), showing that lysozyme in the electrospun fibers is susceptible to heat denaturation under more extreme conditions.

### Example 9

### In Vitro Activity of Disclosed Protein-containing Mucoadhesive Patches

Over 600 different species of microbes reside commensally within the oral cavity in healthy individuals. Many of these organisms have the ability to become pathogenic if the oral mucosa is wounded or compromised, or if there is significant dysbiosis in the microbial flora. Lysozyme is a glycoside hydrolase and acts as an antimicrobial agent by catalyzing the hydrolysis of linkages between N-acetylmuramic acid and N-acetyl-D-glucosamine residues in peptidoglycan, the major cell wall component of Gram-positive bacteria.

The goal of the present study was to evaluate if fiber-released lysozyme was able to cleave peptidoglycan and cause bacterial cell lysis in Streptococcus ratti, a Gram-positive oral bacterium found in the oral cavity that has been associated with dental biofilms and is known to be affected by lysozyme activity. Lysozyme was eluted from electrospun fibers and its effects on inhibiting bacteria growth assessed.

Procedure

Streptococcus ratti (NCTC 10920, Public Health England, Salisbury, UK) was grown overnight in 10 mL brain heart infusion broth (BHI) at 37° C., 5% CO₂ before diluting to OD₆₀₀ of 0.1 in BHI. Samples of electrospun patches (50±0.1 mg) were immersed in 1 mL sterile PBS for 24 h with vortexing for 30 s after immersing and before sampling the eluate. Sterile PBS was used as a negative control and 0.5 mg/mL lysozyme in PBS as a positive control. Additionally, placebo patch samples were eluted in a stock 0.5 mg/mL lysozyme solution. A 12-well plate was filled with BHI (0.5 mL per well) before adding eluate and controls in triplicate (0.5 mL). Each well was inoculated with 0.1 mL bacteria and the plate incubated at 37° C. with OD600 measured every 10 minutes for 15 h with shaking for 1 minute before each reading.

### Results

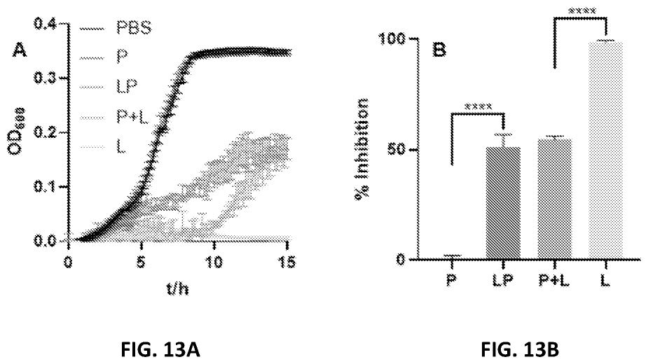

FIG. 13A shows the growth curve of S. ratti measured by optical density at 600 nm over time in the presence of PBS, eluent from placebo fibers (P), eluent from fibers containing lysozyme (LP), placebo fibers eluted in stock lysozyme solution (P+L), and a lysozyme stock solution (L). As shown in FIG. 8B, eluted lysozyme significantly inhibited the growth of S. ratti by 51% after 15 h compared to eluate from placebo fibers control (51.1±5.7 for LP vs 0.6±1.6 for P, p<0.0001). These data clearly show that lysozyme released by electrospun fibers retains its biological activity and is able to inhibit bacterial growth. The disclosed fibers may therefore be effective at treating oral bacterial infections or as covering for oral wounds or lesions that may be susceptible to bacterial infection.

Interestingly, the eluate of the lysozyme electrospun fibers contained 0.43±0.07 mg/mL protein content and caused 51% bacterial inhibition, whereas purified lysozyme of a similar protein concentration (0.5 mg/mL) almost completely abolished S. ratti growth over the same time period (FIG. 13B, L, p<0.0001). To investigate the cause of this apparent reduction in activity, samples of placebo electrospun fibers were eluted in 0.5 mg/mL lysozyme solution (P+L). The resulting eluate inhibited growth to the same extent as the lysozyme electrospun fiber eluent, suggesting that this disparity related to polymer in the sample and not loss of activity during electrospinning. This may be a result of water soluble PVP leaching from the fibers and inhibiting lysozyme activity or encapsulating and protecting the bacteria.

### Example 10

### Evaluating Transmucosal Insulin Delivery From A Disclosed Patch

Procedure

Electrospun patches containing 5% w/w insulin and controls (insulin solution −1 mg/mL in pH 6.8 PBS; placebo patch+artificial saliva in pH 6.8 PBS; and PBS) are applied to tissue engineered epithelial models, e.g., an FNB6 model, (keratinocytes only). To determine the transmucosal delivery of insulin over time, the concentration of insulin in the lower chamber of the well is measured by ELISA by taking aliquots from the wells over an 8 h period, diluting (e.g., in 1% BSA), and freezing before the ELISA assay is performed. After 8 h, the models are washed with PBS and fixed (e.g., with 10% formalin) and the distribution of insulin in the epithelium will be visualized using immunofluorescence.

Results

The amount of insulin delivered transmucosally across the epithelium is measured, in addition to the amount of insulin delivered locally, i.e., inside the epithelium.

As described herein, the polypeptide-containing patches of the present disclosure enabled the encapsulation of an enzyme into polymer fibers with superior encapsulation efficiency and biological activity preservation compared to what has previously been accomplished with uniaxial electrospinning. The approach described herein is considerably simpler to scale up to an industrial manufacturing setting than the frequently reported emulsion and coaxial electrospinning techniques, with fewer parameters to optimize and control. In addition, the disclosed fibers released the majority of the protein in a single burst at a rate appropriate for drug delivery to the oral mucosa. Furthermore, the protein was homogenously distributed, and data suggest that the dual-layer patches maintained desirable mucoadhesive properties. The biological activity of the encapsulated protein was further demonstrated by the inhibition of growth of an oral bacterial strain. This illustrates that the patches are capable of local delivery of an effective amount of therapeutic polypeptides directly to mucosa (e.g., oral mucosa) for the treatment of various diseases and disorders or as a non-invasive method for systemic drug delivery.

The invention is further described by the following numbered embodiments:
1. A polypeptide-containing patch comprising:
   (a) an electrospun fiber layer, wherein the electrospun fibers comprise:
      one or more polymers, and
      a therapeutically effective amount of a therapeutic polypeptide; and
   (b) an impermeable backing layer.
2. The patch of embodiment 1, wherein the electrospun fibers are uniaxial.
3. The patch of any one of embodiments 1-2, wherein the electrospun fiber layer comprises about 0.01% to about 50.0% (wt./wt. %) of the therapeutic polypeptide.
4. The patch of embodiment 3, wherein the electrospun fiber layer comprises about 1.0% to about 5.0% (wt./wt. %) of the therapeutic polypeptide.
5. The patch of any one of embodiments 1-4, wherein the patch provides a therapeutically effective amount of the therapeutic polypeptide for at least about 4 h following application to a patient in need thereof. 6. The patch of any one of embodiments 1-6, wherein the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.
7. The patch of any one of embodiments 1-6, wherein the one or more polymers comprise:
(a) polyvinylpyrrolidone and an ammonio methacrylate copolymer;
(b) polyvinylpyrrolidone and polyethylene glycol;
(c) polyvinylpyrrolidone and polyethylene oxide;
(d) polyvinylpyrrolidone and dextran;
(e) polyvinylpyrrolidone and gelatin;
(f) polyvinylpyrrolidone and polyvinyl alcohol;
(g) polyvinylpyrrolidone, an ammonio methacrylate copolymer and polyethylene glycol;
(h) polyvinylpyrrolidone, an ammonio methacrylate copolymer and polyethylene oxide;
(i) polyvinylpyrrolidone, an ammonio methacrylate copolymer and dextran;
(j) polyvinylpyrrolidone, an ammonio methacrylate copolymer and gelatin; or
(k) polyvinylpyrrolidone, an ammonio methacrylate copolymer and polyvinyl alcohol.
8. The patch of any one of embodiments 1-7, wherein the backing layer is hydrophobic.
9. The patch of embodiment 8, wherein the hydrophobic backing layer comprises polycaprolactone.
10. The patch of any one of embodiments 1-9, wherein the therapeutic polypeptide is selected from the group consisting of a monoclonal antibody, a fragment antigen-binding (Fab fragment) antibody, a single-chain variable fragment (scFv), a full antigen, a fragmented antigen, a protein, a peptide, and an enzyme.
11. The patch of embodiment 10, wherein the therapeutic polypeptide is a monoclonal antibody.
12. The patch of embodiment 11, wherein the monoclonal antibody is selected from the group consisting of loncastuximab, balstilimab, ansuvimab, bimekizumab, omburtamab, tralokinumab, evinacumab, sutimlimab, aducanumab, teplizumab, dostarlimab, margetuximab, naxitamab, inolimomab, oportuzumab, narsoplimab, belantamab, tafasitamab, satralizumab, inebilizumab, sacituzumab, teprotumumab, isatuximab, eptinezumab, trastuzumab, enfortumab, crizanlizumab, brolucizumab, polatuzumab, risankizumab, romosozumab, caplacizumab, ravulizumab, emapalumab, cemiplimab, fremanezumab, moxetumomab, galcanezumab, lanadelumab, mogamuizumab, erenumab, tildrakizumab, ibalizumab, burosumab, durvalumab, emicizumab, benralizumab, ocrelizumab, guselkumab, inotuzumab, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alicrocumab, mepolizumab, evolocumab, dinutuximab, secukinumab, nivolumab, blinatumomab, pembrolizumab, ramucirumab, vedolizumab, siltuximab, ustekinumab, obinutuzumab, certolizumab pegol, ranibizumab, abcixumab, raxibacumab, pertuzumab, brentuximab, belimumab, ipilimumab, denosumab, tocilizumab, ofatumumab, canakinumab, golimumab, ustekinumab, catumaxomab, eculizumab, panitumumab, natalizumab, bevacizumab, cetuximab, efalizumab, omalizumab, tositumomab, ibritumomab, ibritumomab, adalimumab, alemtuzumab, gemtuzumab, infliximab, palivizumab, basilixumab, daclizumab, rituximab, edrecolomab, nebacumab, and muromonab-CD₃.
13. The patch of embodiment 10, wherein the therapeutic polypeptide is a Fab fragment antibody.
14. The patch of embodiment 13, wherein the Fab fragment antibody is selected from the group consisting of certolizumab pegol, ranibizumab, abcixumab, abrezekimab, citatuzumab, lampalizumab, onartuzumab, tadocizumab, arcitumomab, sulesomab, and imiciromab.
15. The patch of embodiment 10, wherein the therapeutic polypeptide is a single-chain variable fragment (scFv).
16. The patch of embodiment 15, wherein the scFV is selected from the group consisting of brolucizumab, duvortuxizumab, efungumab, flotetuzumab, ganitumab, letolizumab, oportuzumab monatox, pexelizumab, and vobarilizumab.
17. The patch of embodiment 10, wherein the therapeutic polypeptide is an antigen.
18. The patch of embodiment 17, wherein the antigen is a full antigen.
19. The patch of embodiment 17, wherein the antigen is a fragmented antigen.
20. The patch of embodiment 17, wherein the antigen is selected from the group consisting of insulin, proinsulin, insulin peptides, GAD65, HSP60, myelin peptides, glatiramer acetate, dnaJP1, APL-1, P140, IPP-201101, NPL001, NPL002, and NPL003.
21. The patch of embodiment 10, wherein the therapeutic polypeptide is a protein.
22. The patch of embodiment 21, wherein the protein is selected from the group consisting of insulin, corticotropin, secretin, growth hormone GH, pegvisoman, mecasermin, factor VIII, factor IX, erythropoietin, filgrastim, oprelvekin, human follicle-stimulating hormone (FSH), human chorionic gonadotropin (HCG), palifermin, becaplermin, lepirudin, anakinra, interleukin 2 (IL2), interferons, peginterferon, etanercept, alefacept, abatacept, rilonacept, romiplostim, and belatacept.
23. The patch of embodiment 10, wherein the therapeutic polypeptide is a peptide.
24. The patch of embodiment 23, wherein the peptide is selected from the group consisting of bradykinin, bivalirudin, buserelin, enfuvirtide, eptifibatide, glatiramer, gramicidin D, lepirudin, leuprolide, liraglutide, lucinactant, octreotide, exenatide, nesiritide, oxytocin, pramlintide, salmon calcitonin, sermorelin, teduglutide, and thymalfasin.
25. The patch of embodiment 10, wherein the therapeutic polypeptide is an enzyme.
26. The patch of embodiment 25, wherein the enzyme is selected from the group consisting of collagenase, β-glucocerebrosidase, alglucosidase-α, laronidase, idursulfase, galsulfase, agalsidase-β, lactase, pancreatic enzymes, adenosine deaminase, tissue plasminogen activator (tPA), factor VIIa, trypsin, botulinum toxin, papain, L-asparaginase, rasburicase, and streptokinase.
27. The patch of any one of embodiments 1-26, wherein the electrospun fibers further comprise an absorption enhancer.
28. The patch of embodiment 27, wherein the absorption enhancer is selected from the group consisting of a fatty acid, a non-ionic surfactant, a polycation, a thiolated polymer, a cyclodextrin, and a cell-penetrating peptide.
29. The patch of embodiment 28, wherein the absorption enhancer is a fatty acid.
30. The patch of embodiment 29, wherein the fatty acid is selected from the group consisting of caproic acid, caprylic acid, capric acid, sodium caprate, lauric acid, stearic acid, oleic acid, linoleic acid, and linolenic acid.
31. The patch of embodiment 28, wherein the absorption enhancer is a non-ionic surfactant.
32. The patch of embodiment 31, the non-ionic surfactant is selected from the group consisting of polysorbate, polyethylene glycol hexadecyl ether, polyoxyethylene alkyl ethers, nonylphenoxypolyoxyethylene (NPPOE), laurate sucrose ester (SE), and sodium glycocholate.
33. The patch of embodiment 28, wherein the absorption enhancer is a polycation.
34. The patch of embodiment 33, wherein the polycation is selected from the group consisting of a chitosan and its quaternary ammonium derivatives, a poly-L-arginine, an aminated gelatin, and cetylpyridinium chloride.
35. The patch of embodiment 28, wherein the absorption enhancer is a thiolated polymer.
36. The patch of embodiment 35, wherein the thiolated polymer is selected from the group consisting of carboxymethyl cellulose-cysteine, polycarbophil (PCP)-cysteine, chitosan-thiobutylamidine, chitosan-thioglycolic acid, chitosan-thioethylamidine, chitosan iminothiolane, chitosan-glutathione conjugates, polyacrylic acid-cysteine, polymethacrylic acid-cysteine, and hyaluronicacid-L-cysteine.
37. The patch of embodiment 28, wherein the absorption enhancer is a cyclodextrin.
38. The patch of embodiment 37, wherein the cyclodextrin is selected from the group consisting of a methylated β-cyclodextrin, hydroxypropyl-β-cyclodextrin, and sulphobutylether-β-cyclodextrin.
39. The patch of embodiment 28, wherein the absorption enhancer is a cell-penetrating peptide.
40. The patch of embodiment 39, wherein the cell-penetrating peptide is selected from the group consisting of penetratin, Tat (YGRKKKRRQRRR), R6, R8, R9, pVEC (LLIILRRRIRKQAHAHSK), RRL helix (RRLRRLLRRLRRLLRRLR), shuffle (RWFKIQMQIRRWKNKK), and penetramax (KWFKIQMQIRRWKNKR) . 41. The patch of any one of embodiments 1-40, wherein the electrospun fiber layer further comprises a therapeutically effective amount of an additional therapeutic agent.
42. The patch of embodiment 41, wherein the additional therapeutic agent is a steroid.
43. A method of treating a condition in a patient in need thereof, the method comprising administering a patch of any one of embodiments 1-42, wherein the condition is selected from the group consisting of recurrent aphthous stomatitis, oral lichen planus, pemphigoid, pemphigus, oral mucositis, Graft versus host disease (GvHD), Bechet's disease, lupus erythematosus, vulva lichen planus, Lipschutz ulcers, lichen simplex chronicus, vulva psoriasis, allergies, autoimmune disorders or disorders with an immune component (multiple sclerosis, rheumatoid arthritis), osteoporosis, inflammatory bowel disease, hypercholesterolemia, asthma, hematologic malignancies, solid tumors, allograft rejection, and infectious organisms.
44. The method of embodiment 43, wherein the patch is administered once a day.
45. The method of embodiment 43, wherein the patch is administered twice a day.
46. The method of embodiment 43, wherein the patch is administered three times a day.
47. The method of any one of embodiments 43-46, wherein the patch is administered for at least a week.
48. The method of any one of embodiments 43-47, wherein the patch is administered for about 2-4 weeks.
49. The method of any one embodiments 43-48, wherein the patch is administered for at least a month.
50. A process for preparing a patch of any one of embodiments 1-49, the process comprising:
   (a) combining an aqueous alcoholic solvent with one or more polymers and a therapeutic polypeptide to provide an electrospinning mixture;
   (b) electrospinning the Step (a) mixture to provide electrospun fibers; and
   (c) attaching an impermeable backing layer to the Step (b) electrospun fibers.
51. The process of embodiment 50, wherein the conductivity of the electrospinning mixture of Step (a) is from about 150 μS/cm to about 600 μS/cm.
52. The process of embodiment 51, wherein the conductivity of the electrospinning mixture of Step (a) is from about 150 μS/cm to about 300 μS/cm.
53. The process of any one of embodiments 50-52, wherein the aqueous alcoholic solvent comprises ethanol and water.
54. The process of embodiment 53, wherein the solvent comprises about 40% to about 97% (vol./vol. %) of ethanol.
55. The process of embodiment 54, wherein the solvent comprises about 80% to about 97% (vol./vol. %) of ethanol.
56. The process of any one of embodiments 50-55, wherein the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.
57. The process of embodiment 56, wherein the one or more polymers comprise:
   (a) polyvinylpyrrolidone and an ammonio methacrylate copolymer;
   (b) polyvinylpyrrolidone and polyethylene glycol;
   (c) polyvinylpyrrolidone and polyethylene oxide;
   (d) polyvinylpyrrolidone and dextran;
   (e) polyvinylpyrrolidone and gelatin;
   (f) polyvinylpyrrolidone and polyvinyl alcohol;
   (g) polyvinylpyrrolidone, an ammonio methacrylate copolymer and polyethylene glycol;
   (h) polyvinylpyrrolidone, an ammonio methacrylate copolymer and polyethylene oxide;
   (i) polyvinylpyrrolidone, an ammonio methacrylate copolymer and dextran;
   (j) polyvinylpyrrolidone, an ammonio methacrylate copolymer and gelatin; or
   (k) polyvinylpyrrolidone, an ammonio methacrylate copolymer and polyvinyl alcohol.

What is claimed is:

1. A polypeptide-containing patch comprising:
(a) an electrospun fiber layer, wherein the electrospun fibers comprise:
one or more polymers, and
a therapeutically effective amount of a therapeutic polypeptide; and
(b) an impermeable backing layer.

2. The patch of claim 1, wherein the electrospun fibers are uniaxial.

3. The patch of any one of claims 1-2, wherein the electrospun fiber layer comprises about 0.01% to about 50.0% (wt./wt. %) of the therapeutic polypeptide.

4. The patch of claim 3, wherein the electrospun fiber layer comprises about 1.0% to about 5.0% (wt./wt. %) of the therapeutic polypeptide.

5. The patch of any one of claims 1-4, wherein the patch provides a therapeutically effective amount of the therapeutic polypeptide for at least about 4 h following application to a patient in need thereof.

6. The patch of any one of claims 1-6, wherein the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

7. The patch of any one of claims 1-6, wherein the one or more polymers comprise:
(a) polyvinylpyrrolidone and an ammonio methacrylate copolymer;
(b) polyvinylpyrrolidone and polyethylene glycol;
(c) polyvinylpyrrolidone and polyethylene oxide;
(d) polyvinylpyrrolidone and dextran;
(e) polyvinylpyrrolidone and gelatin;
(f) polyvinylpyrrolidone and polyvinyl alcohol;
(g) polyvinylpyrrolidone, an ammonio methacrylate copolymer and polyethylene glycol;
(h) polyvinylpyrrolidone, an ammonio methacrylate copolymer and polyethylene oxide;
(i) polyvinylpyrrolidone, an ammonio methacrylate copolymer and dextran;
(j) polyvinylpyrrolidone, an ammonio methacrylate copolymer and gelatin; or
(k) polyvinylpyrrolidone, an ammonio methacrylate copolymer and polyvinyl alcohol.

8. The patch of any one of claims 1-7, wherein the backing layer is hydrophobic.

9. The patch of claim 8, wherein the hydrophobic backing layer comprises polycaprolactone.

10. The patch of any one of claims 1-9, wherein the therapeutic polypeptide is selected from the group consisting of a monoclonal antibody, a fragment antigen-binding (Fab fragment) antibody, a single-chain variable fragment (scFv), a full antigen, a fragmented antigen, a protein, a peptide, and an enzyme.

11. The patch of claim 10, wherein the therapeutic polypeptide is a monoclonal antibody.

12. The patch of claim 11, wherein the monoclonal antibody is selected from the group consisting of loncastuximab, balstilimab, ansuvimab, bimekizumab, omburtamab, tralokinumab, evinacumab, sutirnlimab, aducanumab, teplizumab, dostarlimab, margetuximab, naxitamab, inolimomab, oportuzumab, narsoplimab, belantamab, tafasitamab, satralizurnab, inebilizumab, sacituzumab, teprotumumab, isatuximab, eptinezumab, trastuzumab, enfortumab, crizanlizumab, brolucizumab, polatuzumab, risankizumab, romosozumab, caplacizumab, ravulizumab, emapalumab, cemiplimab, fremanezumab, moxetumomab, galcanezumab, lanadelumab, mogamuizumab, erenumab, tildrakizumab, ibalizumab, burosumab, durvalumab, emicizumab, benralizumab, ocrelizumab, guselkumab, inotuzumab, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alicrocumab, mepolizumab, evolocumab, dinutuximab, secukinumab, nivolumab, blinatumomab, pembrolizumab, ramucirumab, vedolizumab, siltuximab, ustekinumab, obinutuzumab, certolizumab pegol, ranibizumab, abcixumab, raxibacumab, pertuzumab, brentuximab, belimumab, ipilimumab, denosumab, tocilizumab, ofatumumab, canakinumab, golimumab, ustekinumab, catumaxomab, eculizumab, panitumumab, natalizumab, bevacizumab, cetuximab, efalizumab, omalizumab, tositumomab, ibritumomab, ibritumomab, adalimumab, alemtuzumab, gemtuzumab, infliximab, palivizumab, basilixumab, daclizumab, rituximab, edrecolomab, nebacumab, and muromonab-CD₃.

13. The patch of claim 10, wherein the therapeutic polypeptide is a Fab fragment antibody.

14. The patch of claim 13, wherein the Fab fragment antibody is selected from the group consisting of certolizumab pegol, ranibizumab, abcixumab, abrezekimab, citatuzumab, lampalizumab, onartuzumab, tadocizumab, arcitumomab, sulesomab, and imiciromab.

15. The patch of claim 10, wherein the therapeutic polypeptide is a single-chain variable fragment (scFv).

16. The patch of claim 15, wherein the scFV is selected from the group consisting of brolucizumab, duvortuxizumab, efungumab, flotetuzumab, ganitumab, letolizumab, oportuzumab monatox, pexelizumab, vobarilizumab.

17. The patch of claim 10, wherein the therapeutic polypeptide is an antigen.

18. The patch of claim 17, wherein the antigen is a full antigen.

19. The patch of claim 17, wherein the antigen is a fragmented antigen.

20. The patch of claim 17, wherein the antigen is selected from the group consisting of insulin, proinsulin, insulin peptides, GAD65, HSP60, myelin peptides, glatiramer acetate, dnaJP1, APL-1, P140, IPP-201101, NPL001, NPL002, and NPL003.

21. The patch of claim 10, wherein the therapeutic polypeptide is a protein.

22. The patch of claim 21, wherein the protein is selected from the group consisting of insulin, corticotropin, secretin, growth hormone GH, pegvisoman, mecasermin, factor VIII, factor IX, erythropoietin, filgrastim, oprelvekin, human follicle-stimulating hormone (FSH), human chorionic gonadotropin (HCG), palifermin, becaplermin, lepirudin, anakinra, interleukin 2 (IL2), interferons, peginterferon, etanercept, alefacept, abatacept, rilonacept, romiplostim, and belatacept.

23. The patch of claim 10, wherein the therapeutic polypeptide is a peptide.

24. The patch of claim 23, wherein the peptide is selected from the group consisting of bradykinin, bivalirudin, buserelin, enfiivirtide, eptifibatide, glatiramer, gramicidin D, lepirudin, leuprolide, liraglutide, lucinactant, octreotide, exenatide, nesiritide, oxytocin, pramlintide, salmon calcitonin, se) norelin, teduglutide, and thymalfasin.

25. The patch of claim 10, wherein the therapeutic polypeptide is an enzyme.

26. The patch of claim 25, wherein the enzyme is selected from the group consisting of collagenase, β-glucocerebrosidase, alglucosidase-α, laronidase, idursulfase, galsulfase, agalsidase-β, lactase, pancreatic enzymes, adenosine deaminase, tissue plasminogen activator (tPA), factor VIIa, trypsin, botulinum toxin, papain, L-asparaginase, rasburicase, and streptokinase.

27. The patch of any one of claims 1-26, wherein the electrospun fibers further comprise an absorption enhancer.

28. The patch of claim 27, wherein the absorption enhancer is selected from the group consisting of a fatty acid, a non-ionic surfactant, a polycation, a thiolated polymer, a cyclodextrin, and a cell-penetrating peptide.

29. The patch of claim 28, wherein the absorption enhancer is a fatty acid.

30. The patch of claim 29, wherein the fatty acid is selected from the group consisting of caproic acid, caprylic acid, capric acid, sodium caprate, lauric acid, stearic acid, oleic acid, linoleic acid, and linolenic acid.

31. The patch of claim 28, wherein the absorption enhancer is a non-ionic surfactant.

32. The patch of claim 31, the non-ionic surfactant is selected from the group consisting of polysorbate, polyethylene glycol hexadecyl ether, polyoxyethylene alkyl ethers, Nonylphenoxypolyoxyethylene (NPPOE), laurate sucrose ester (SE), and sodium glycocholate.

33. The patch of claim 28, wherein the absorption enhancer is a polycation.

34. The patch of claim 33, wherein the polycation is selected from the group consisting of a chitosan and its quaternary ammonium derivatives, a poly-L-arginine, an aminated gelatin, and cetylpyridinium chloride.

35. The patch of claim 28, wherein the absorption enhancer is a thiolated polymer.

36. The patch of claim 35, wherein the thiolated polymer is selected from the group consisting of carboxymethyl cellulose-cysteine, polycarbophil (PCP)-cysteine, chitosan-thiobutylamidine, chitosan-thioglycolic acid, chitosan-thioethylamidine, chitosan iminothiolane, chitosan-glutathione conjugates, polyacrylic acid-cysteine, polymethacrylic acid-cysteine, and hyaluronicacid-L-cysteine.

37. The patch of claim 28, wherein the absorption enhancer is a cyclodextrin.

38. The patch of claim 37, wherein the cyclodextrin is selected from the group consisting of a methylated β-cyclodextrin, hydroxypropyl-β-cyclodextrin, and sulphobutylether-β-cyclodextrin.

39. The patch of claim 28, wherein the absorption enhancer is a cell-penetrating peptide.

40. The patch of claim 39, wherein the cell-penetrating peptide is selected from the group consisting of penetratin, Tat (YGRKKKRRQRRR), R6, R8, R9, pVEC (LLIILRRRIRKQAHAHSK), RRL helix (RRLRRLLRRLRRLLRRLR), shuffle (RWFKIQMQIRRWKNKK), and penetramax (KWFKIQMQIRRWKNKR).

41. A method of treating a condition in a patient in need thereof, the method comprising administering a patch of any one of claims 1-40, wherein the condition is selected from the group consisting of recurrent aphthous stomatitis, oral lichen planus, pemphigoid, pemphigus, oral mucositis, Graft versus host disease (GvHD), Bechet's disease, lupus erythematosus, vulva lichen planus, Lipschutz ulcers, lichen simplex chronicus, vulva psoriasis, allergies, autoimmune disorders or disorders with an immune component (multiple sclerosis, rheumatoid arthritis), osteoporosis, inflammatory bowel disease, hypercholesterolernia, asthma, hematologic malignancies, solid tumors, allograft rejection, and infectious organisms.

42. The method of claim 41, wherein the patch is administered once a day.

43. The method of claim 41, wherein the patch is administered twice a day.

44. The method of claim 41, wherein the patch is administered three times a day.

45. The method of any one of claims 41-44, wherein the patch is administered for at least a week.

46. The method of any one of claims 41-45, wherein the patch is administered for about 2-4 weeks.

47. The method of any one claims 41-46, wherein the patch is administered for at least a month.

48. A process for preparing a patch of any one of claims 1-47, the process comprising:
(a) combining an aqueous alcoholic solvent with one or more polymers and a therapeutic polypeptide to provide an electrospinning mixture;
(b) electrospinning the Step (a) mixture to provide electrospun fibers; and
(c) attaching an impermeable backing layer to the Step (b) electrospun fibers.

49. The process of claim 48, wherein the conductivity of the electrospinning mixture of Step (a) is from about 150 μS/cm to about 600 μS/cm.

50. The process of claim 49, wherein the conductivity of the electrospinning mixture of Step (a) is from about 150 μS/cm to about 300 μS/cm.

51. The process of any one of claims 48-50, wherein the aqueous alcoholic solvent comprises ethanol and water.

52. The process of claim 51, wherein the solvent comprises about 40% to about 97% (vol./vol. %) of ethanol.

53. The process of claim 52, wherein the solvent comprises about 80% to about 97% (vol./vol. %) of ethanol.

54. The process of any one of claims 48-53, wherein the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

55. The process of claim 54, wherein the one or more polymers comprise:
(a) polyvinylpyrrolidone and an ammonio methacrylate copolymer;
(b) polyvinylpyrrolidone and polyethylene glycol;
(c) polyvinylpyrrolidone and polyethylene oxide;
(d) polyvinylpyrrolidone and dextran:
(e) polyvinylpyrrolidone and gelatin;
(f) polyvinylpyrrolidone and polyvinyl alcohol;
(g) polyvinylpyrrolidone, an ammonio methacrylate copolymer and polyethylene glycol;
(h) polyvinylpyrrolidone, an ammonio methacrylate copolymer and polyethylene oxide;
(i) polyvinylpyrrolidone, an ammonio methacrylate copolymer and dextran;
(j) polyvinylpyrrolidone, an ammonio methacrylate copolymer and gelatin; or
(k) polyvinylpyrrolidone, an ammonio methacrylate copolymer and polyvinyl alcohol.
